(19) United States
(12) Patent Application Publication
(10) Pub. No.: US 2025/0058024 A1
(43) Pub. Date: Feb. 20, 2025

(54) INJECTABLE TOUGH AND VISCOELASTIC BTA HYDROGEL

(71) Applicants: UNIVERSITEIT MAASTRICHT, Maastricht (NL); ACADEMISCH ZIEKENHUIS MAASTRICHT, Maastricht (NL)

(72) Inventors: Matthew Brandon BAKER, Maastricht (NL); Shahzad HAFEEZ, Maastricht (NL); Lorenzo MORONI, Maastricht (NL); Ana Agustina ALDANA, Maastricht (NL)

(21) Appl. No.: 18/706,336

(22) PCT Filed: Nov. 1, 2022
(86) PCT No.: PCT/EP2022/080447

§ 371 Date: Apr. 30, 2024

(30) Foreign Application Priority Data
Nov. 1, 2021 (EP) 21205815.0

(51) Int. Cl. A61L 27/52; A61L 27/18; A61L 27/38; B33Y 10/00; B33Y 70/00; C07C 69/76; C07C 205/57; C07D 207/46; C08G 65/00

(52) U.S. Cl. CPC A61L 27/52; A61L 27/18; A61L 27/38; B33Y 10/00; B33Y 70/00; C07C 69/76; C07C 205/57; C07D 207/46; C08G 65/00

(57) ABSTRACT

The invention describes benzene-1,3,5-tricarboxamide (BTA) based hydrogels. The hydrogels have purpose in tissue culturing for in vitro applications such as drug screening or in vivo applications such as transplantation. The hydrogels are particularly tough but still allow 3D printing and cell culturing. The invention further describes 3D printing of the hydrogels and uses of the hydrogels.

### FIELD OF THE INVENTION

The invention relates to hydrogels. Particularly the invention relates to BTA based supramolecular or crosslinked hydrogels which are suitable for 3D printing and culturing cells. The hydrogels may be used for drug screening, cell culture matrices, or for transplanting as load bearing tissue in a subject. Then invention further relates to methods of 3D printing or bioprinting the hydrogel

### INTRODUCTION

Living tissues such as cartilage, muscle, tendon and ligaments exhibit high elasticity, toughness, and fast recovery. Superior mechanical properties are often attributed to collagen hierarchical fibril structure across multiple lengths scales in the tissues. Collagen fibrils are composed of a self-assembled structure of tropocollagen (TC) molecules mainly driven by non-covalent interactions and steric stabilization. These fibrils are further stabilized by intramolecular and intermolecular covalent cross-links (via aldol condensation).

Hydrogels are a class of materials that mimics many aspects of soft tissues and offer the potential to be used as instructive biomaterials and structural implants. However, covalently cross-linked hydrogels are generally brittle and supramolecular hydrogels are generally soft and offer low resistance to loading. Many efforts have been devoted to enhancing the toughness and strength of hydrogels. Tough hydrogels have been made by forming a double network, dual cross-linking of strong covalent bonds and physical cross-links. Physical cross-links are reversible and responsible for energy dissipation. Tough hydrogels based on conjoined networks, Hofmeister effect-assisted hydrophobic aggregation of polymer chains, and by creating crystalline domains to deflect a crack have also been developed. For most of the above strategies, the creation of tough hydrogels often involves toxic chemicals, day's long polymerization reactions, which hampered application in cell culture and bioprinting applications. Double network tough hydrogels using natural and synthetic polymers have been developed which are cyto-compatible. Most of the hydrogels however have homogenous mesh-like structures, lack anisotropic fibril structure, offer partial recovery after large deformation, and translation to bioinks has remain limited. Nature has utilized extensively one dimensional supramolecular polymers as powerful structural motifs such as tropocollagen, microtubules, and actin filament for strong and tough tissues. Scientist have been only able to realize recently specific and directional properties of supramolecular polymers and started to design 1D structural motifs, short peptides, peptide amphiphile (PA) and 2-ureido-4 [1H]-pyrimidinone (UPy) are few examples. Tough hydrogels with tuneable mechanical properties are designed using UPy structural motifs and toughness in short peptide and PA remains largely unexplored. Some recent studies engineered shear-thinning behaviour in short self-assembled peptides based bioinks; however, investigation of shear-thinning, self-healing properties in UPy and PA remains largely unexplored. Using host-guest supramolecular interactions toughness has been engineered in hydrogels, however, a bioink with engineered toughness and fibril morphology that can be bioprinted into complex 3D life-like structures and allow tuning stiffness and mechanical properties still remains highly desirable.

Three dimensional (3D) printing and bioprinting has emerged as promising technology which offers rapid prototyping and enables the creation of complex tissue-like structures such as heart valve, vascular networks, corneal dome, kidney using hydrogel. Rheological properties of most of the bioinks developed are poorly suited for extrusion bioprinting and layer by layer assembly since most of them are highly viscous liquids, can block nozzle owing to fast UV cross-linking, involve large pH shifts for collagen gels, and supraphysiological levels of calcium ions for alginate bioinks. Traditional bioinks do not offer shear-thinning properties which are required to prevent cell membrane damage from excessive shear stresses during injectability and extrusion bioprinting. In traditional bioinks, shear-thinning properties can be introduced by mixing two different polymers such as mixing gelatine with methacrylate hyaluronic acid (HAMA). Bioprinted filament can be stabilised by secondary cross-links such as methyacrylation. Various reversible chemistries including dynamic covalent bonds supramolecular host-guest interactions, hydrophobic interactions, and hydrogen bonding interactions have been utilized for introducing shear-thinning properties. However, filament collapse and shape retention still remain a challenge.

Synthetic bioinks are elastic, fragile, and still require engineering of toughness in fibrillar structure found in load bearing living tissues.

For example WO2021041372 describes a method for bioprinting a bioink using a biopolymer backbone with grafted thereto a biorthogonal chemistry group. WO2018200944 and Leenders et al. (Mater. Horiz. 2014, 1, 116) describe benzene-1,3,5-tricarboxamide (BTA) based supramolecular polymers, which are not particularly amenable to crosslinking reactions under physiological conditions.

Therefore there is a continues need for improved hydrogels that allow 3D printing which can be used to replace or support load bearing tissues such as cartilage, muscle, and tendon in patients. Particularly there is a need for providing improved hydrogels that are amenable to crosslinking reaction under physiological conditions. These objectives, among others, are addressed by the invention as defined in the appended claims.

### SUMMARY OF THE INVENTION

In a first aspect the invention relates to a compound having the formula represented by formula (Ia):

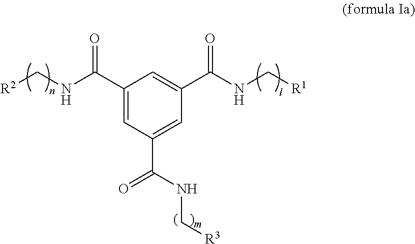

wherein:
each I, m, and n are individually an integer between 1 to 18; and
R¹, R² and R³ are each individually selected from a polymer or a reactive group, wherein
the polymer is represented by

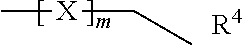

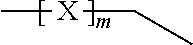

wherein represents,

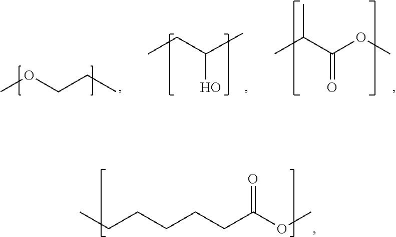

polyester co-polymers, polysaccharides, polyacrylates, polyacrylamides, polyurethanes or co-polymers thereof, wherein m is an integer from 1 to 20000 and
wherein R⁴ is a natural chain end or a reactive group, and
wherein the polymer is selected such that the compound is water soluble or water dispersible; and
wherein the reactive group is selected from the group consisting of:
   a C₁-C₃₆ linear or branched alkyl or alkenyl or alkynyl or cycloalkyl,

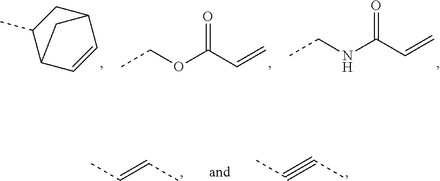

azides, alkynes, amines, oximes, hydrazides, semicarbazides, aldehydes, strained triple bonds, epoxides, malimides, furans or substitutions thereof, preferably substitutions with a linear or branched alkyl group, acrylate or acrylamide or sulfides thereof, and wherein at least one of R¹, R² or R³ is a reactive group.

When used herein a natural chain end should be interpreted as a group selected from H, —OH, —NH₂, a linear or branched C₁-C₆ alkyl group, acrylate or acrylamide or sulfides thereof.

In a second aspect the invention relates to a hydrogel comprising the compound of the first aspect of the invention.

In a third aspect the invention relates to a method for 3D printing a structure, the method comprising:
a. printing a composition comprising a compound having the formula represented by formula (Ia):

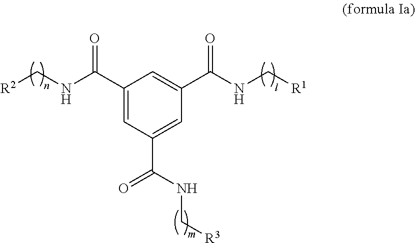

wherein:
each I, m, and n are individually an integer between 1 to 18; and
R¹, R² and R³ are each individually selected from a polymer or a reactive group, wherein
the polymer is represented by

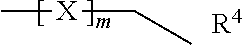

wherein

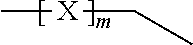

represents,

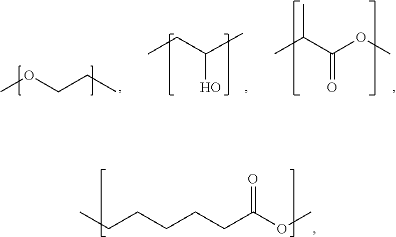

polyester co-polymers, polysaccharides, polyacrylates, polyacrylamides, polyurethanes or co-polymers thereof, wherein m is an integer from 1 to 20000 and
wherein R⁴ is a natural chain end or a reactive group, and
wherein the polymer is selected such that the compound is water soluble or water dispersible; and
wherein the reactive group is selected from the group consisting of:
   a C₁-C₃₆ linear or branched alkyl or alkenyl or alkynyl or cycloalkyl,

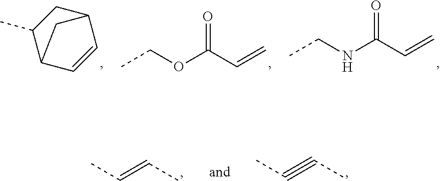

azides, alkynes, amines, oximes, hydrazides, semicarbazides, aldehydes, strained triple bonds, epoxides, malimides, furans or substitutions thereof, preferably substitutions with a linear or branched alkyl group, acrylate or acrylamide or sulphides thereof, and wherein at least one of R¹, R² or R³ is a reactive group.

In a fourth aspect the invention relates to the 3D printed structure obtained or obtainable by the method according to the third aspect of the invention. In a fifth aspect the invention relates to use of the hydrogel according to the second aspect of the invention or the 3D printed structure according to the fourth aspect of the invention as a tissue model, extracellular matrix analog or in drug testing. In a sixth aspect the invention relates to the hydrogel according to the second aspect of the invention or the 3D printed structure according to the fourth aspect of the invention for use in a surgical method, the method comprising implanting the hydrogel or the 3D printed structure in a subject in need thereof.

### BRIEF DESCRIPTION OF THE FIGURES

FIGS. 1. A) and B) Molecular structure of several exemplary BTA structures and C) cross-linkers employed in the study.

FIG. 2. Schematic of BTA hydrogelator and hydrogels network structure before and after cross-linking. A) Cartoon representation of BTA hydrogelator, stacking of BTA molecules to form fiber. B) a schematic of supramolecular gel network. C) Cross-linking of nanofiber in supramolecular hydrogel using C) di-thiol covalent cross-link which is an 8 atoms long short di-thiol cross-link, D) a longer di-thiol cross-linker and E) cross-linking using 4-arm thiol cross-link.

FIG. 3. Self-assembly studies in dilute solutions. A) Nile red fluorescence as a function of norbornene BTA polymeric hydrogelator concentration. B) nile red fluorescence for fixed versus non-fixed solution at 5 mg/ml of BTA hydrogelator. Nanofibers of norbornene BTA at 10 mg/ml for C) supramolecular solution and D) solution exposed to UV light for cross-linking after 24 hours aging E) supramolecular fibers at 5 mg/ml aged for 30 days demonstrating nanofibers with diameter around 5 nm and F) around 15 nm perhaps due to bundling of nanofibers.

FIG. 4: Aligned hierarchical anisotropic fibril morphology before and after cross-linking. A) Optical image of supramolecular bulk hydrogel. A1 shows anisotropic fibril morphology of supramolecular hydrogel images using cryo-SEM and A2 shows zoomed in image of fibril. B) Extruding of supramolecular hydrogel into continuous uniform fiber and cross-linking using short di-thiol in presence of LAP photoinitiator and UV light. Cross-linked fiber is strong and stretchable. C) cryo-SEM images of supramolecular gel after extruding through 22 gauge syringe, and on the right to arrow is an image of extruded hydrogel which was cross-linked using short di-thiol cross-linker.

FIG. 5: Cross-linking of supramolecular hydrogel leads to strong, tough, and stretchable hydrogels. A) Optical images of supramolecular gel under extreme compression (top panel), cutting supramolecular gel using spatula (middle panel), and optical image of cutting of supramolecular hydrogel using sharp blade (bottom panel). B) Optical images of cross-linked hydrogel under extreme compression (top panel), while cutting fixed gel using spatula (middle panel) and cutting of fixed gel using sharp blade (bottom panel). C) Supramolecular gel when compressed with spatula shows pseudo plastic deformation and F) cross-linked hydrogel shows resistance to compression and recover to original length after compression. G) Stretching of extruded supramolecular and H) cross-linked fiber.

FIG. 6: Tuning of mechanical properties of supramolecular and cross-linked hydrogel. A) A1 shows storage moduli (filled symbol) and loss moduli (empty symbol) as a function of angular frequency of supramolecular gel for 2.5% (w/v), 5% (w/v) and 10% (w/v). A2 shows rapid cross-linking of supramolecular gel when exposed to UV light source. A3 shows storage moduli and loss moduli as a function of angular frequency of cross-linked gel. B) storage moduli as a function of angular frequency of supramolecular and fixed gel for different mole equivalent of short di-thiol cross-linker, C) storage modulus as a function of angular frequency for supramolecular and cross-linked gel when hydrogel cross-linked using di-thiol cross-linker of different molecular lengths. C) Frequency sweep of supramolecular gel for 2-arm versus 4-arm. E) stress-strain curve of cross-linked hydrogel during compression test. F) Elastic modulus values calculated from compression test and G) Toughness values calculated from compression test.

FIG. 7: Shear-thinning, self-healing, injectability and 3D printability of BTA hydrogel. A) shear-thinning behavior of hydrogel showing decrease in complex viscosity with increasing shear rate, B) a quick recovery in storage modulus after rupturing gel network showed rapid self-healing behavior supramolecular gel, C) supramolecular hydrogel extruded into BIOMATT logo, rectangle structure, hexane like ring structure and hollow structure. D) Cartilage model printed using supramolecular hydrogel without cross-linking. D1 showed hollow cartilage model and D2, D3 and D4 showed solid 3D printed cartilage model. E) Shows cartilage model which was cross-linked after printing each layer of the hydrogel. E1, E2 and E3 shows different views of cartilage printed model. E4 and E5 shows printing of cartilage model with two BTA formulations with different mechanical properties. D) Panel shows cartilage model recovery after a young man weighing at least 60 kg stood on it.

FIG. 8. Cell viability after injectability and in 3D bioprinted hydrogels A) live-dead images of ATDC5 chondrocytes in supramolecular and cross-linked hydrogel after 2 hours. Green color represses live cell and red color represents dead cell. B) Live-dead images of ATDC5 chondrocytes in after injectability through 22 gauge after 2 hours of injection. C) live-dead images of cartilage model from day 0 until day 11. Cartilage model was printed using 22 gauge and with ATDC encapsulated. D) Graph shows the cell number and cell area of live and dead cells for supramolecular and cross-linked hydrogel.

FIG. 9 broadly defines the molecular structure of the compounds as described herein according to formula Ia.

### DETAILED DESCRIPTION OF THE INVENTION

To simultaneously address the need for rapid shear-thinning, self-healing and tunable toughness in one dimensional fibrillar hydrogel and bioprintability into complex 3D shapes, the inventors attempted to design hydrogels that undergo self-assembly via noncovalent interactions resulting in fibrils, which are stabilized by intrafiber and interfiber cross-links. It was found that supramolecular hydrogels with transient and dynamic interactions are an ideal Bioink if the hydrogel offers shear-thinning and self-healing properties, facilitate mild cell encapsulation, allow the creation of stable 3D life-like structures. In this system in particular, intrafiber and interfiber cross-links can be introduced for stabilization, and tuning strength and toughness. The inventors have approached this by designing and developing a benzene-1,3,5-tricarboxamide (BTA) derived hydrogelator building block, which undergoes self-assembly via 3-fold hydrogen bonding resulting in fibril hydrogel. Owing to the fast reversibility of supramolecular interactions, BTA hydrogel offers rapid shear-thinning, self-healing properties and can be extruded into complex shapes with good shape fidelity. The BTA building block possesses norbornene as an external hydrophobic group that can undergo photoinitiated orthogonal thiol-ene chemistry and BTA fibril can be stabilized with intrafiber and interfiber cross-links for tuning stiffness, strength, and toughness. The subunits are assembled on a stack, which is then cross-linked. Crosslinking can be performed between subunits directly (depicted as A+A reaction), or using a crosslinker (depicted as A+B reaction). Furthermore, it is envisioned that the subunits can have a monomer or dimer conformation.

Therefore, in a first aspect the invention relates to a compound having the formula represented by formula (Ia):

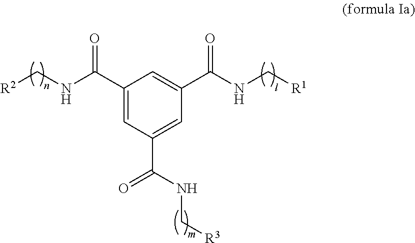

wherein:
each I, m, and n are individually an integer between 1 to 18; and
R¹, R² and R³ are each individually selected from a polymer or a reactive group, wherein
the polymer is represented by

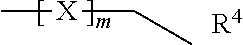

wherein

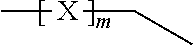

represents,

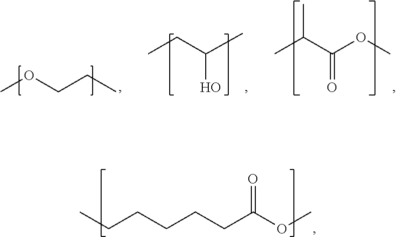

polyester co-polymers, polysaccharides, polyacrylates, polyacrylamides, polyurethanes or co-polymers thereof, wherein m is an integer from 1 to 20000 and
wherein R⁴ is a natural chain end or a reactive group, and
wherein the polymer is selected such that the compound is water soluble or water dispersible; and
wherein the reactive group is selected from the group consisting of:
   a C₁-C₃₆ linear or branched alkyl or alkenyl or alkynyl or cycloalkyl,

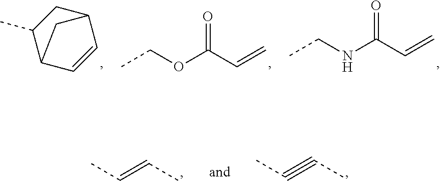

azides, alkynes, amines, oximes, hydrazides, semicarbazides, aldehydes, strained triple bonds, epoxides, malimides, furans or substitutions thereof, preferably substitutions with a linear or branched alkyl group, acrylate or acrylamide or sulfides thereof, and wherein at least one of R¹, R² or R³ is a reactive group. In an embodiment at least one, preferably two or all of the groups R¹, R² and R³ are individually selected form the group consisting of:

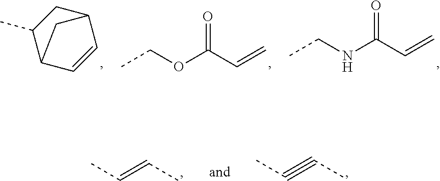

azides, alkynes, amines, oximes, hydrazides, semicarbazides, aldehydes, strained triple bonds, epoxides, malimides, furans or substitutions thereof, preferably substitutions with a linear or branched alkyl group, acrylate or acrylamide or sulfides thereof.

The formula Ia broadly defines monomers, but also encompasses dimers, as for example defined below with Formulas Ib, Iva and IVb.

Thus in an embodiment the invention relates to a compound represented by formula (IVa) or formula (IVb):

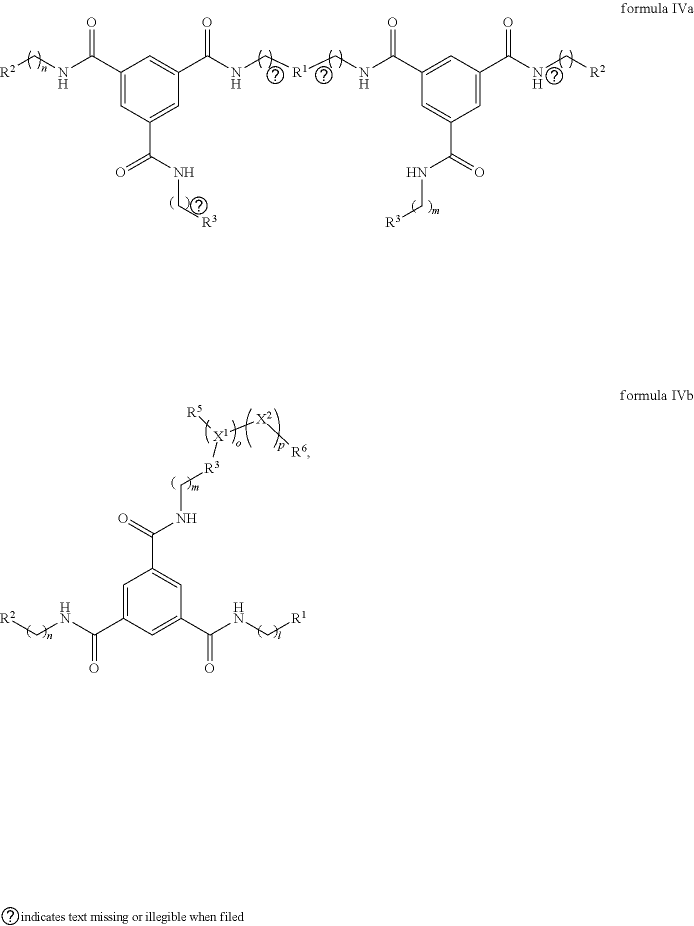

wherein X¹ and X² are each individually selected from:

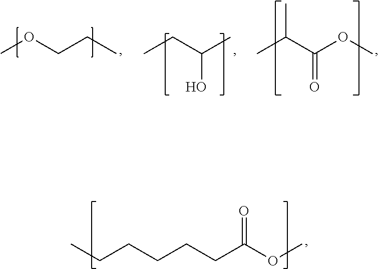

polyester co-polymers, polysaccharides, polyacrylates, polyacrylamides, polyurethanes or co-polymers thereof, wherein o and p are individually an integer from 1 to 20000, and wherein R⁵ and R⁶ are each individually selected from H, OH, NH₂, a C₁-C₃₆ linear or branched alkyl or alkenyl or alkynyl or cycloalkyl,

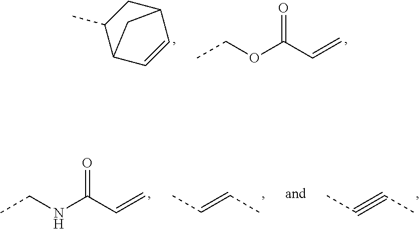

azides, alkynes, amines, oximes, hydrazides, semicarbazides, aldehydes, strained triple bonds, epoxides, malimides, furans or substitutions thereof or

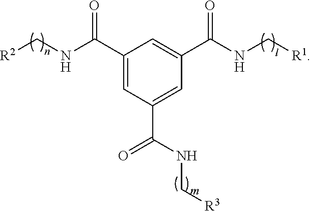

As described above, it is envisioned that stacks are formed of the compounds broadly described herein, which can then be cross-linked. Crosslinking can be performed between reactive groups on the compound or using crosslinkers. Therefore, in case crosslinkers are not envisioned reactive groups should be chosen for at least some of the R. It is understood however that not every needs to be a reactive group and not even every compound need to comprise a reactive groups as they may be tacked with different compounds that do comprise a reactive group. Thus in an embodiment one or more R is selected from azides, alkynes, amines, oximes, hydrazides, semicarbazides, aldehydes, strained triple bonds or substitutions thereof, or the compound is a mixture of related compound and at least some, for example 10, 20, 30, 40, 50, 60, 70, 80, or 90% of the compounds in the mixture comprise at least one R selected from azides, alkynes, amines, oximes, hydrazides, semicarbazides, aldehydes, strained triple bonds or substitutions thereof. In a preferred embodiment the reactive group is a group that is amenable to reactions at physiological conditions. In a preferred embodiment the invention relates to compounds represented by the Formula (Ib):

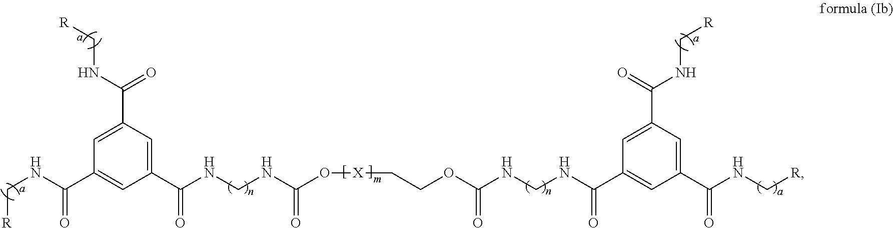

wherein:
each a is individually an integer between 1 to 18;
each n is individually an integer between 6 to 18;
wherein

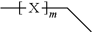

represents,

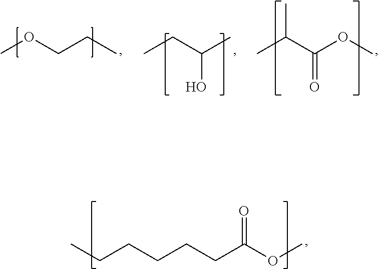

polyester co-polymers, polysaccharides, polyacrylates, polyacrylamides, polyurethanes, or co-polymers thereof, wherein m is an integer from 1 to 20000; and each R is individually selected from the group consisting of:
a C₁-C₃₆ linear or branched alkyl or alkenyl or alkynyl or cycloalkyl,

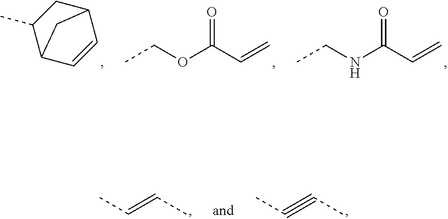

azide, alkyne, amine, oxime, hydrazide, semicarbazide, aldehyde, strained triple bond, epoxide, malimide, furan, or substitutions thereof, preferably substitutions with a linear or branched alkyl group, acrylate or acrylamide or sulfides thereof, with the proviso that at least two, preferably at least three, more preferably all four, R are individually selected form the group consisting of:

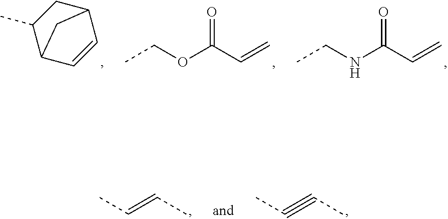

azide, alkyne, amine, oxime, hydrazide, semicarbazide, aldehyde, strained triple bond, epoxide, malimide, furan, or substitutions thereof, preferably substitutions with a linear or branched alkyl group, acrylate or acrylamide or sulfides thereof.

In a further embodiment the invention relates to a compound having the formula represented by formula (Ib):

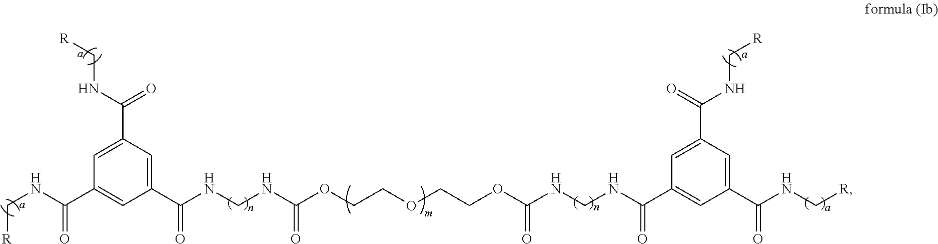

wherein:
each a is individually an integer between 1 to 18;
each n is individually an integer between 6 to 18;
m is an integer from 1 to 20000; and
each R is individually selected from the group consisting of:
a C₁-C₃₆ linear or branched alkyl or alkenyl or alkynyl or cycloalkyl,

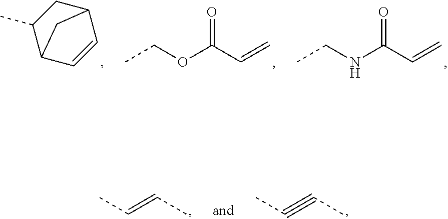

azide, alkyne, amine, oxime, hydrazide, semicarbazide, aldehyde, strained triple bond, epoxide, malimide, furans or substitution thereof, preferably substitutions with a linear or branched alkyl group, acrylate or acrylamide or sulphides thereof, with the proviso that at least two, preferably at least three, more preferably all four, R are individually selected form the group consisting of:

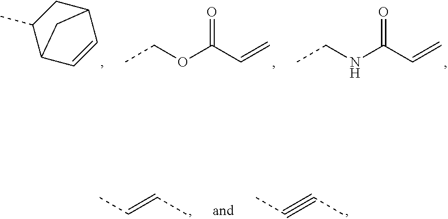

or substitutions thereof, preferably substitutions with a linear or branched alkyl group, acrylate or acrylamide or sulphides thereof.

In a further embodiment the invention relates to compound having the formula represented by formula (IIa) or (Ilb):

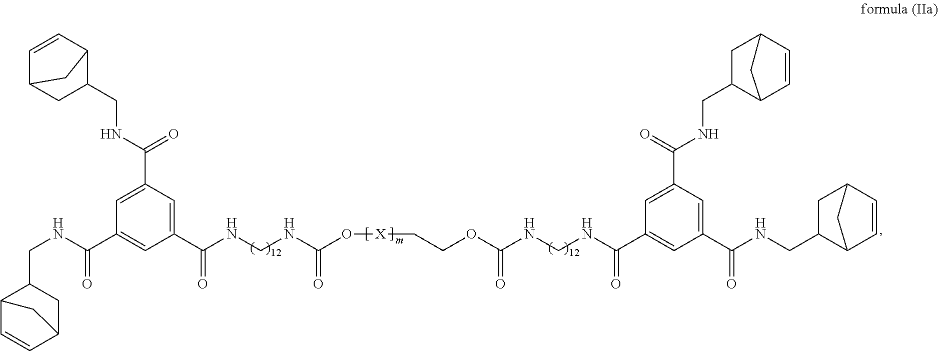

wherein

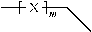

represents,

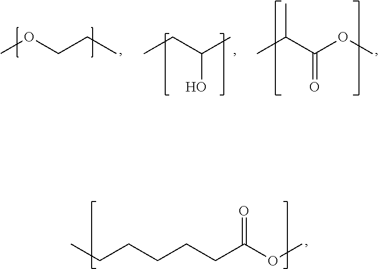

polyester co-polymers, polysaccharides, polyacrylates, polyacrylamides, polyurethanes, or co-polymers thereof, wherein m is an integer from 1 to 20000;

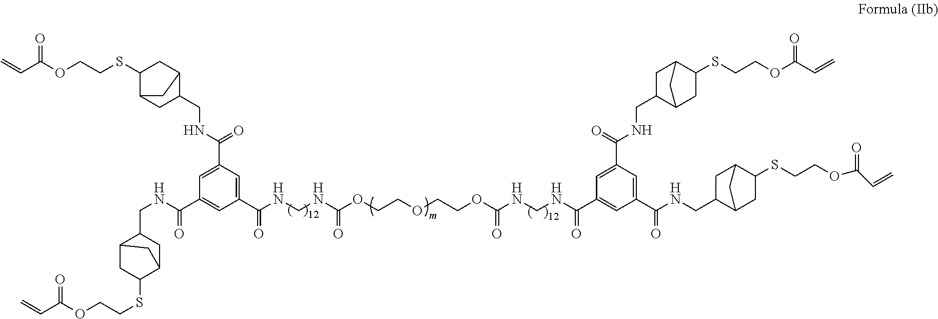

wherein:
each n is individually an integer between 6 to 18; and m is an integer from 1 to 20000. The BTA compound according to formula I can be synthesized using known synthesizing routes. An exemplary synthesizing route is described in Example 2 below. Briefly summarized the synthesis can be performed as described in reaction schemes 1 and 2 below:

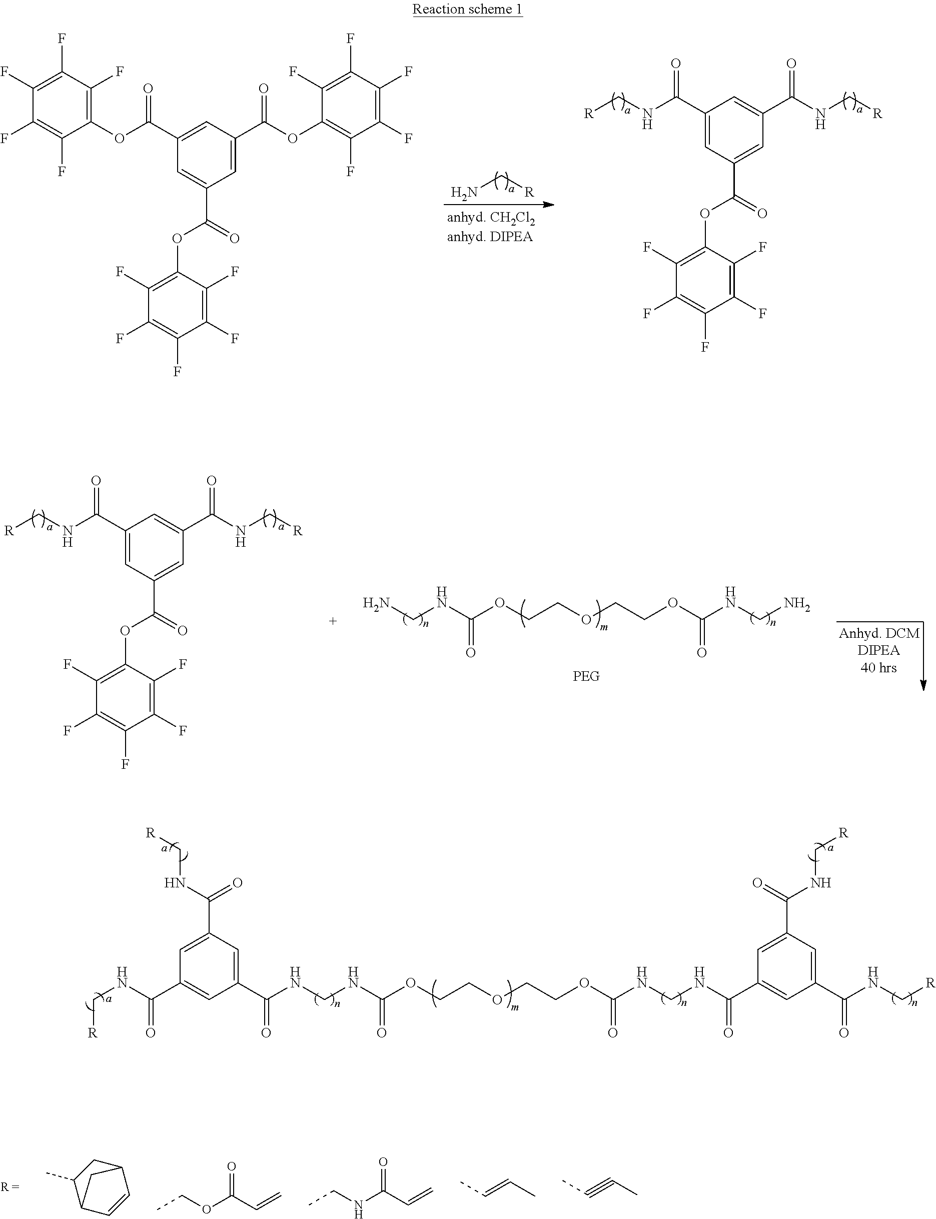

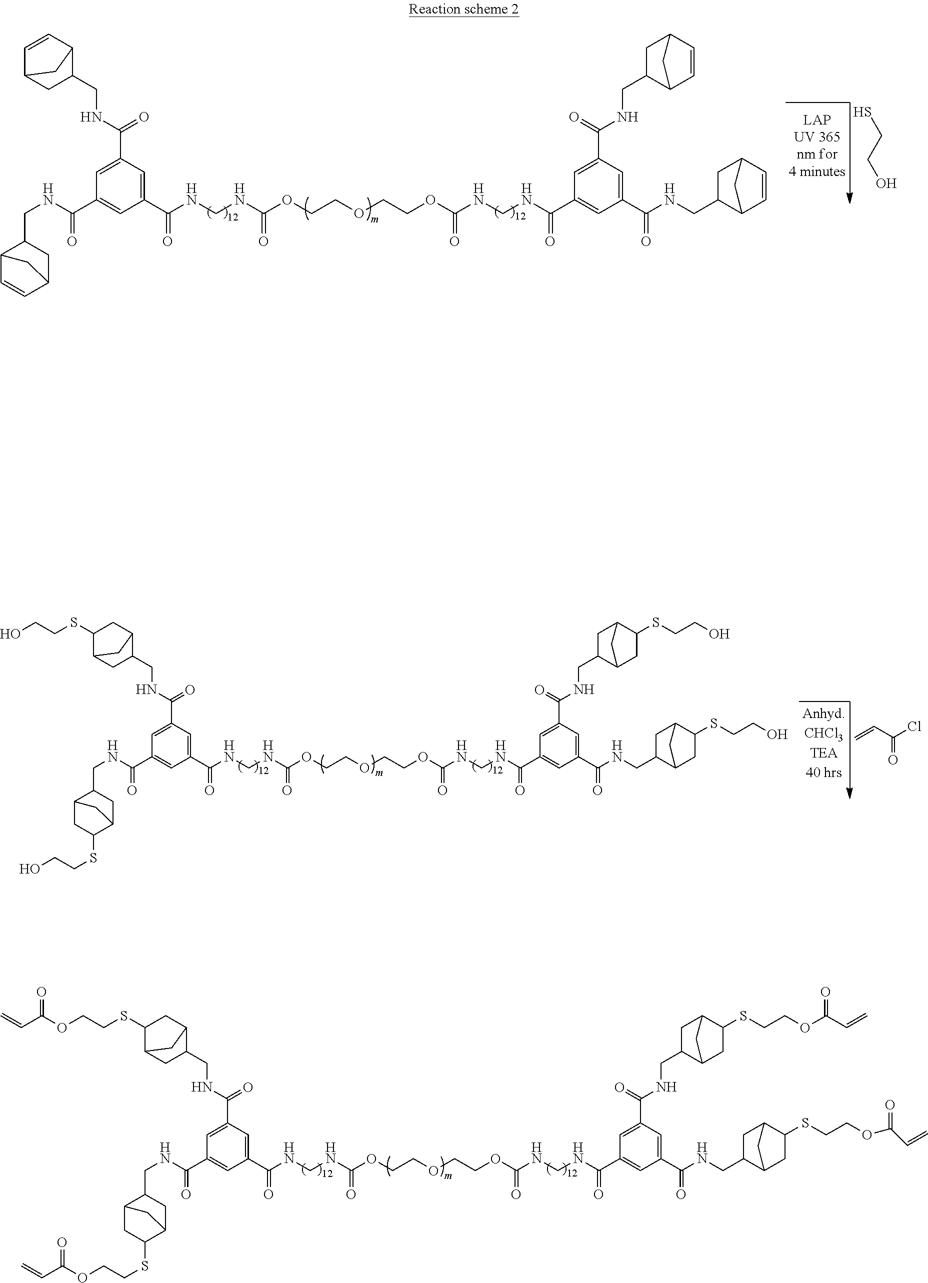

The functional groups R allow crosslinking of the compound to create a stabilized gel. It is noted that the functional groups (R) may be individually selected and thus do not need to be identical. The functional groups are preferably chosen such that they allow crosslinking with a crosslinking agent or direct crosslinking with either a second compound molecule or itself. Therefore preferable 1, 2, 3 or 4 of the functional groups R are selected form the group consisting of:

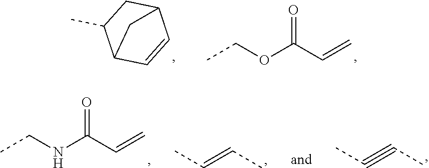

or substitutions thereof, preferably substitutions with a linear or branched alkyl group, acrylate or acrylamide or sulfides thereof. More preferably 1, 2, 3 or 4 of the functional groups R are selected form the group consisting of:

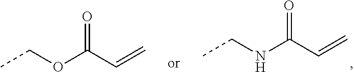

or substitutions thereof, preferably substitutions with a linear or branched alkyl group, acrylate or acrylamide or sulphides thereof. Particularly preferred options are an acrylate or an acrylamide, as it eliminates the need for adding a separate crosslinker. It is further envisioned that one or two of the R functional groups are a C₁-C₃₆ linear or branched alkyl or alkenyl or alkynyl or cycloalkyl, or substitutions thereof, preferably substitutions with a linear or branched alkyl group, acrylate or acrylamide or sulphides thereof (i.e. a functional group that does not allow crosslinking), as it still leaves 2 or 3 R functional groups for crosslinking.

In certain embodiments, the compound is represented by formula (IIa) or (IIb):

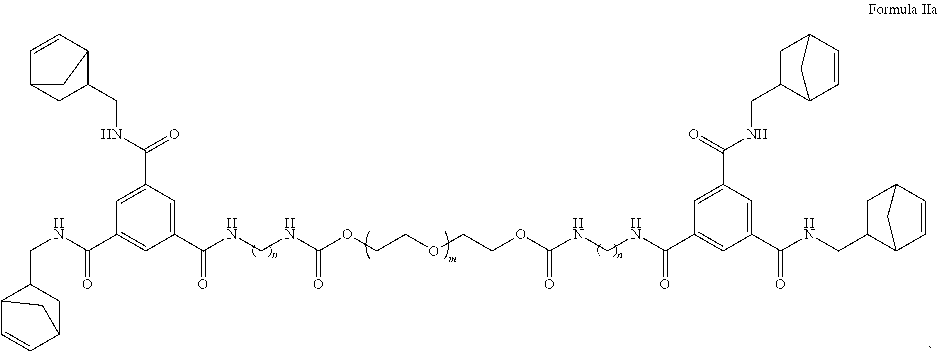

wherein:
each n is individually an integer between 6 to 18; and
m is an integer from 1 to 20000;

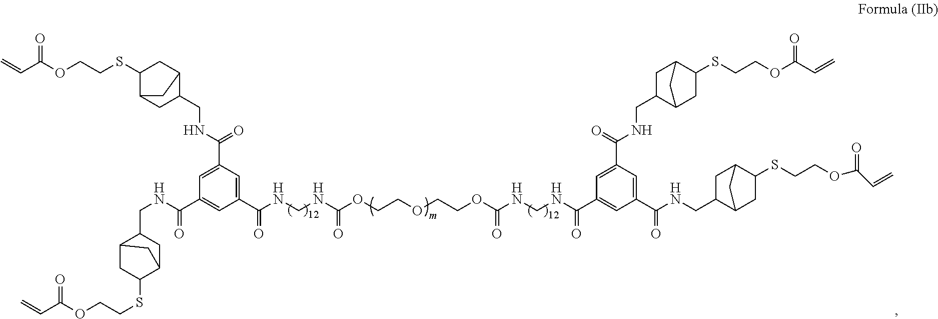

wherein:
each n is individually an integer between 6 to 18; and
m is an integer from 1 to 20000.

The inventors have found that compounds according to formula I have surprisingly beneficial properties to be used as hydrogels as a supramolecular gel, due to the molecule's tendency to form fibril like structures. Further, the hydrogel is found to be biocompatible. In order to increase biocompatibility it is further envisioned that peptides, antibodies, growth factors, DNA strands and/or glycans can be integrated in the compound. Thus in an embodiment the compound is further modified with the covalent attachment of one or more peptides, antibodies, growth factors, DNA strands, glycans or combinations thereof. The one or more peptides may individually comprise 2 or more amino acids, such as for example 2, 3, 4, 5, 6, 7, 8, 9, 10, 11, 12, 13, 14, 15, 16, 17, 18, 19, or 20 or more amino acids.

Therefore, in a further aspect the invention relates to a hydrogel comprising the compound according to the first aspect of the invention.

It was further found that particularly tough hydrogels can be formed when the compound is crosslinked. Crosslinking may be done directly using a compound where one or more of the R functional groups is an acrylate, a vinyl group, or acrylamide, or indirectly by using a crosslinking agent able to crosslink with one or more of the R functional groups of the compound. Therefore, in an embodiment the hydrogel further comprises a cross-linking agent. In an embodiment the crosslinking agent is a compound comprising two or more reactive groups. In an embodiment the crosslinking agent is a compound comprising two or more reactive groups each individually selected from the group consisting of azide, alkyne, amine, oxime, hydrazide, thiol, furan, malimide, acrylate, amine and derivatives thereof. In an embodiment the crosslinker is defined as:

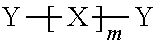

wherein

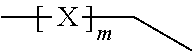

represents

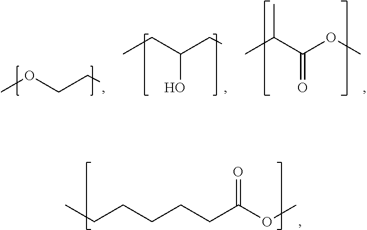

polyester co-polymers, polysaccharides, polyacrylates, polyacrylamides, polyurethanes or co-polymers thereof, wherein m is an integer from 1 to 20000, and wherein each Y is individually selected from an azide, alkyne, amine, oxime, hydrazide, thiol, furan, malimide, acrylate, amine or a derivative thereof. In an embodiment the crosslinking agent is represented by formula (IIIa), formula (IIIb), formula (IIIc), formula (IIId):

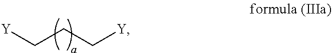

wherein a is an integer ranging from 0 to 12;

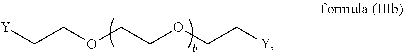

wherein b is an integer ranging from 0 to 10000;

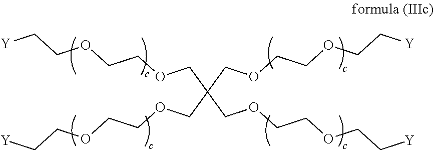

wherein each c is individually an integer ranging from 0 to 250; or

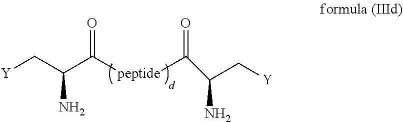

wherein

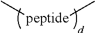

represents a peptide of 2 to 45 amino acids, preferably an enzymatically cleavable peptide; or

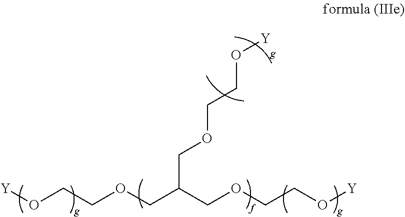

wherein f is an integer ranging from 1 to 10 and wherein each g is individually an integer ranging from 0 to 250
and wherein in the formulas Illa, Illb, Illc, Illd and Ille each Y individually is selected from an azide, alkyne, amine, oxime, hydrazide, thiol, furan, malimide, acrylate, amine or a derivative thereof. In an embodiment, the crosslinking agent is or comprises a compound comprising two or more thiol groups. In an embodiment, the crosslinking agent is represented by formula (Va), formula (Vb), formula (Vc), formula (Vd), or formula (Ve):

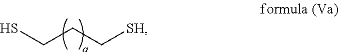

wherein a is an integer ranging from 0 to 12;

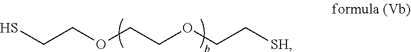

wherein b is an integer ranging from 0 to 10000;

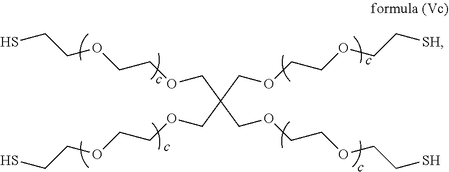

wherein each c is individually an integer ranging from 0 to 250; or

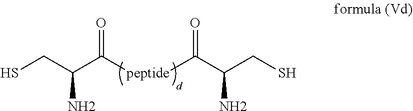

wherein

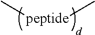

represents a peptide of 2 to 45 amino acids, preferably an enzymatically cleavable peptide; or

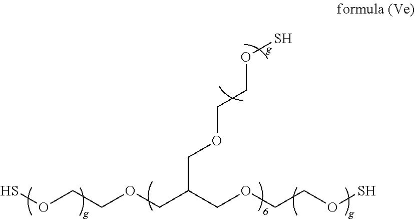

wherein each g is individually an integer ranging from 0 to 250.

Crosslinking may be done using light and can be performed with the presence or absence of a photoinitiator. Conditions that may be used are light with a wave length between 200 and 600 nm e.g. around 400 nm, where a light intensity of 0.1 mW/cm² to 100 mW/cm² is used for 5 seconds to 10 minutes, however the invention is not limited to these parameters. The compositions may further comprise a photointiator such as for example lithium phenyl-2,4,6-trimethylbenzoylphosphinate (LAP), but other suitable photoinitiators are known to the skilled person. Preferably the photoinitiator is a water soluble photoinitiator. For example LAP may be used in a concentration range of 0.05 to 20 mM. Presence of a photoinitiator is not mandatory for crosslinking, but may be preferably in for example 3D printing applications. In another embodiment the crosslinking agent may be an azide, alkyne, amine, oxime, hydrazide, or a derivative thereof. For example the crosslinker may be a polymer defined as

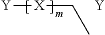

wherein

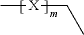

represents,

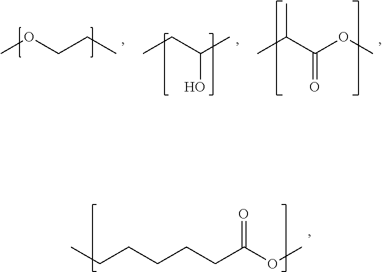

polyester co-polymers, polysaccharides, or co-polymers thereof, wherein m is an integer from 1 to 20000, and wherein each Y is individually selected from an azide, alkyne, amine, oxime, hydrazide, or a derivative thereof. The stiffness of the hydrogel is among others determined by the amount of polymer present. Therefore in an embodiment the hydrogel comprises between 0.25 to 30 w/v %, preferably between 1 and 10 w/v %, of the compound, for example at least, 0.25, 0.5, 0.75, 1, 2, 3, 4, 5 w/v % and/or less than 10, 9, 8, 7, 6 or 5 w/v % of the compound. The stiffness of the hydrogel is further determined by the ratio of crosslinking agent to the compound. Therefore, in an embodiment the molar ratio of the compound to cross-linking agent is between 10:1 and 1:4, for example 10:1, 9:1, 8:1, 7:1, 6:1, 5:1, 4:1, 3:1, 2:1, 1:1, 1:2, 1:3 or 1:4.

It is further envisioned that cells can be embedded in the hydrogel. For example, when the hydrogel is intended to be used as replacement for cartilage, chondrocytes may be embedded to further the biological relevance of the hydrogel. Therefore, in an embodiment, the hydrogel further comprises cells, cell aggregates or organoids, preferably wherein the cells, cell aggregates or organoids are selected from chondrocytes, human umbilical vein endothelial cells (HUVECs), myocytes, neuronal cells, kidney cells, stem cells or kidney organoids.

A particular advantage of the hydrogel as described herein is that it can be printed to provide tailor made 3D printed objects. Therefore, in an aspect the invention relates to a method for 3D printing a structure, the method comprising:
a. printing a composition comprising a compound as broadly defined in the first aspect of the invention.

In an embodiment the invention relates to a method for 3D printing a structure, the method comprising:
a. printing a composition comprising a compound having the formula represented by formula (Ia):

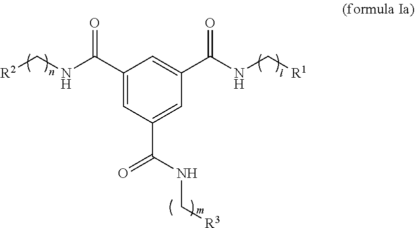

wherein:
each I, m, and n are individually an integer between 1 to 18; and
R¹, R² and R³ are each individually selected from a polymer or a reactive group, wherein
the polymer is represented by

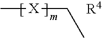

wherein

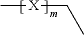

represents,

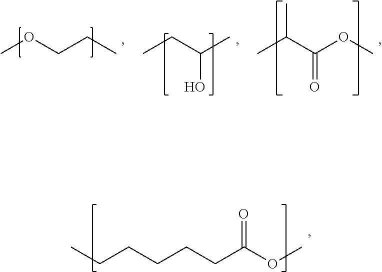

polyester co-polymers, polysaccharides, polyacrylates, polyacrylamides, polyurethanes or co-polymers thereof, wherein m is an integer from 1 to 20000 and
wherein R⁴ is a natural chain end or a reactive group, and
wherein the polymer is selected such that the compound is water soluble or water dispersible; and
wherein the reactive group is selected from the group consisting of:

a C₁-C₃₆ linear or branched alkyl or alkenyl or alkynyl or cycloalkyl,

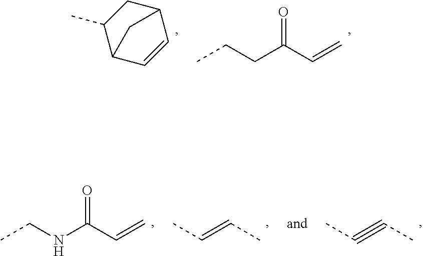

azides, alkynes, amines, oximes, hyrazides, semicarbazides, aldehydes, strained triple bonds, epoxides, malimides, furans or substitutions thereof, preferably substitutions with a linear or branched alkyl group, acrylate or acrylamide or sulfides thereof, and wherein at least one of R¹, R² or R³ is a reactive group.

In an embodiment the invention relates to a method for 3D printing a structure, the method comprising:
a. printing a composition comprising a compound represented by formula (IVa) or formula (IVb):

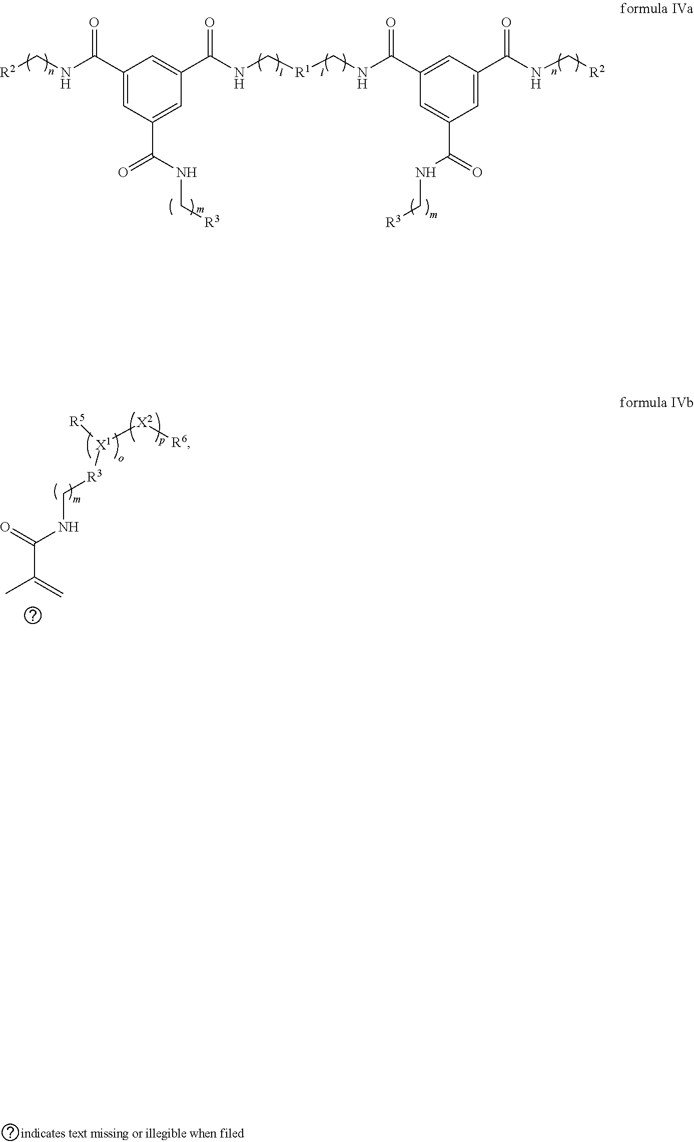

wherein X¹ and X² are each individually selected from:

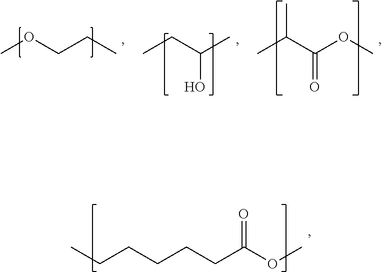

polyester co-polymers, polysaccharides, polyacrylates, polyacrylamides, polyurethanes or co-polymers thereof, wherein o and p are individually an integer from 1 to 20000, and wherein R⁵ and R⁶ are each individually selected from H, OH, NH₂, a C₁-C₃₆ linear or branched alkyl or alkenyl or alkynyl or cycloalkyl,

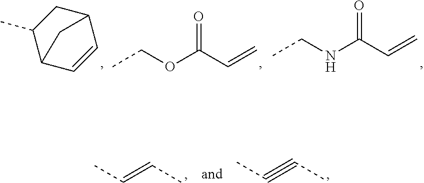

azides, alkynes, amines, oximes, hydrazides, semicarbazides, aldehydes, strained triple bonds, epoxides, malimides, furans or substitutions thereof or

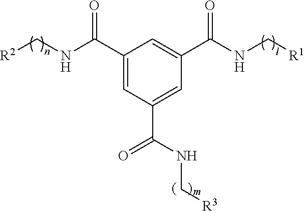

In an embodiment the invention relates to a method for 3D printing a structure, the method comprising:
a. printing a composition comprising a compound having the formula represented by formula (Ib):

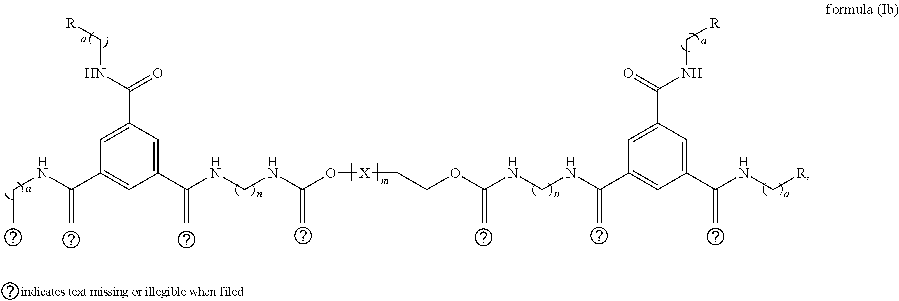

wherein:
each a is individually an integer between 1 to 18;
each n is individually an integer between 6 to 18;
wherein

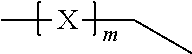

represents,

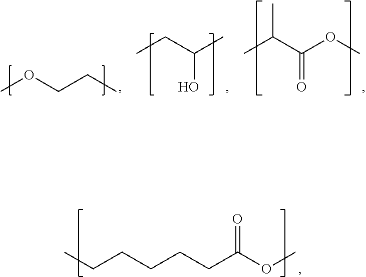

polyester co-polymers, polysaccharides, polyacrylates, polyacrylamides, polyurethanes, or co-polymers thereof, wherein m is an integer from 1 to 20000; and
each R is individually selected from the group consisting of:
a C₁-C₃₆ linear or branched alkyl or alkenyl or alkynyl or cycloalkyl,

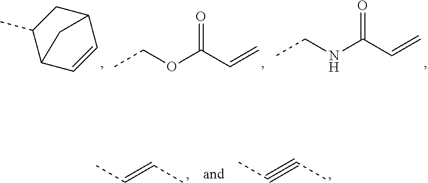

azides, alkynes, amines, oximes, hydrazides, semicarbazides, aldehydes, strained triple bonds, epoxides, malimides, furans, or substitutions thereof, preferably substitutions with a linear or branched alkyl group, acrylate or acrylamide or sulfides thereof, with the proviso that at least two, preferably at least three, more preferably all four, R are individually selected form the group consisting of:

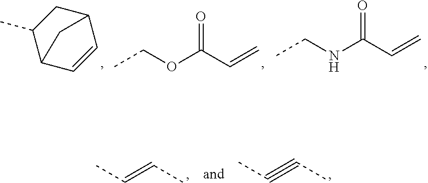

azides, alkynes, amines, oximes, hydrazides, semicarbazides, aldehydes, strained triple bonds, epoxides, malimides, furans, or substitutions thereof, preferably substitutions with a linear or branched alkyl group, acrylate or acrylamide or sulfides thereof.

In an embodiment the compound is represented by formula (Ia):

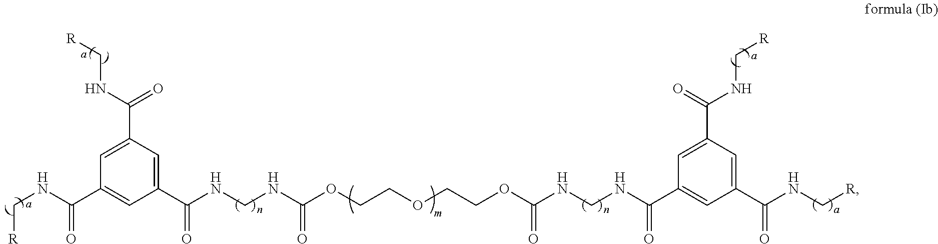

wherein:
each a is individually an integer between 1 to 18;
each n is individually an integer between 6 to 18;
m is an integer from 1 to 20000; and
each R is individually selected from the group consisting of:
a C₁-C₃₆ linear or branched alkyl or alkenyl or alkynyl or cycloalkyl,

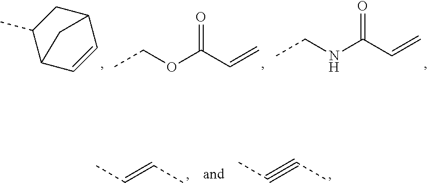

azides, alkynes, amines, oximes, hyrazides, semicarbazides, aldehydes, strained triple bonds, epoxides, malimides, furans, or substitutions thereof, preferably substitutions with a linear or branched alkyl group, acrylate or acrylamide or sulfides thereof, with the proviso that at least two, preferably at least three, more preferably all four, R are individually selected form the group consisting of:

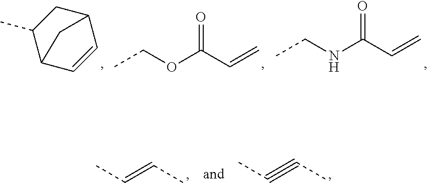

azides, alkynes, amines, oximes, hyrazides, semicarbazides, aldehydes, strained triple bonds, epoxides, malimides, furans, or substitutions thereof, preferably substitutions with a linear or branched alkyl group, acrylate or acrylamide or sulfides thereof. In an embodiment the compound is represented by the formula (Ib):

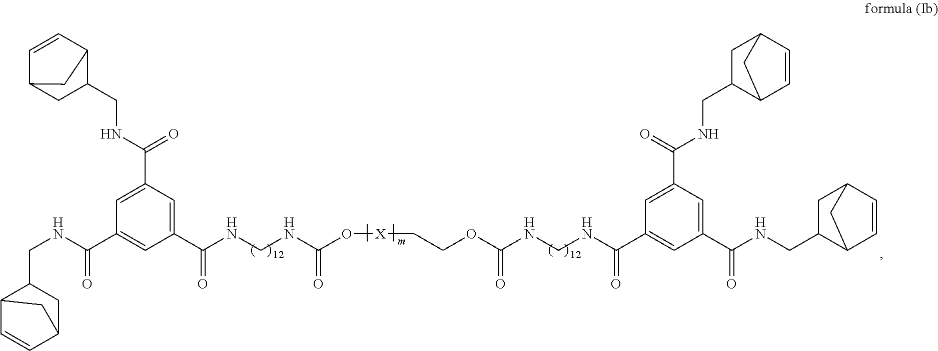

wherein

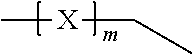

represents,

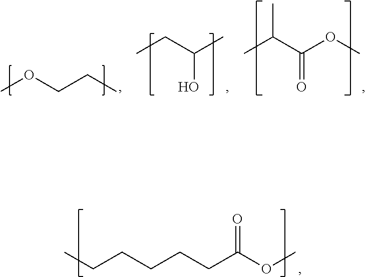

polyester co-polymers, polysaccharides, polyacrylates, polyacrylamides, polyurethanes, or co-polymers thereof, wherein m is an integer from 1 to 20000.

In an embodiment the compound is represented by formula (IIa) or (IIb):

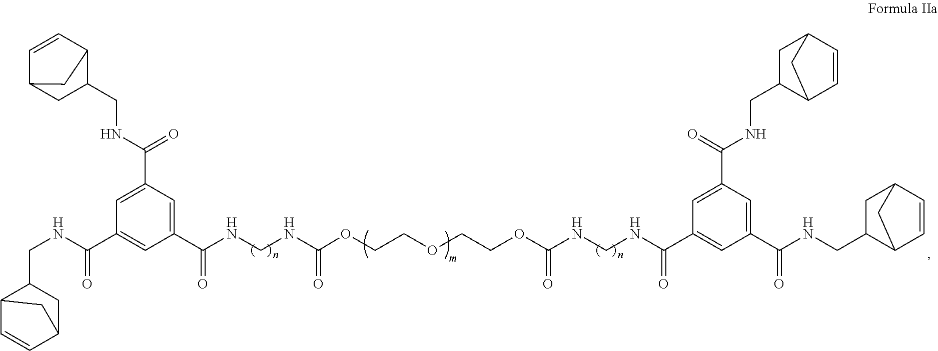

wherein:
each n is individually an integer between 6 to 18; and
m is an integer from 1 to 20000;

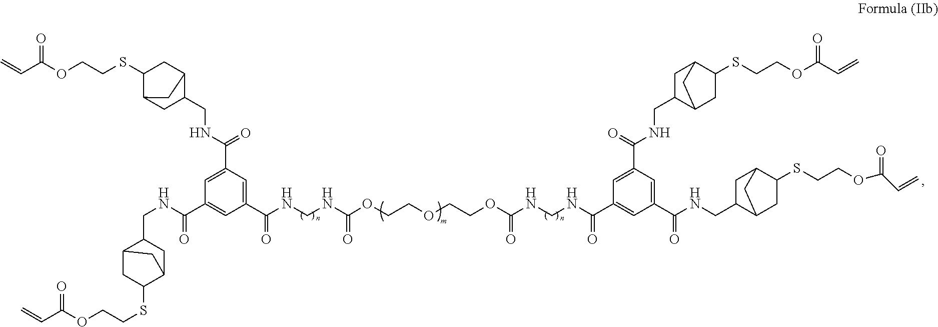

wherein:
each n is individually an integer between 6 to 18; and
m is an integer from 1 to 20000.

In an embodiment of the method, the composition further comprises a cross-linking agent.

In an embodiment of the method the crosslinking agent is or comprises a compound comprising two or more thiol groups, preferably wherein the crosslinking agent is represented by formula (Va), formula (Vb), formula (Vc), formula (Vd), or formula (Ve):

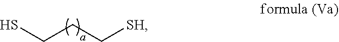

wherein a is an integer ranging from 0 to 12;

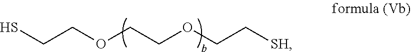

wherein b is an integer ranging from 0 to 10000;

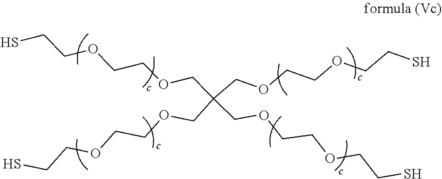

wherein each c is individually an integer ranging from 0 to 250; or

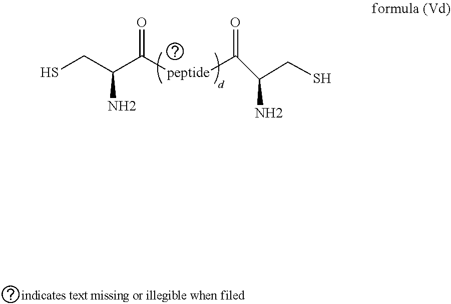

wherein

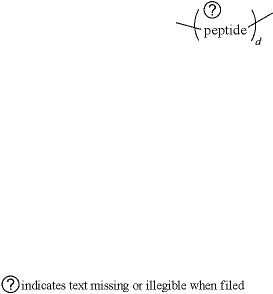

represents a peptide of 2 to 45 amino acids, preferably an enzymatically cleavable peptide; or

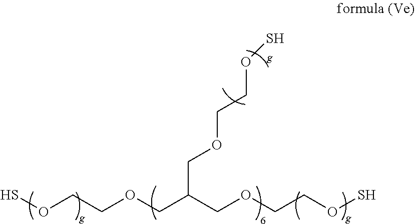

wherein each g is individually an integer ranging from 0 to 250; and wherein the method further comprises the step:
b. cross-linking the supramolecular hydrogel structure during or after printing the structure to obtain a cross-linked hydrogel structure.

Preferably the crosslinking is performed using light as described herein.

In a further preferred embodiment the composition further comprises a photoinitiator, preferably a water soluble photoinitiator. A non-limiting example of a suitable photoinitiator is LAP. Adding a photoinitiator ensures rapid onset of the crosslinking process, which is beneficial in 3D printing applications.

In an embodiment the crosslinking agent is an azide, alkyne, amine, oxime, hydrazide, thiol, furan, malimide, acrylate, amine or a derivative thereof.

In an embodiment the composition wherein the composition further comprises cells, cell aggregates or organoids, preferably wherein the cells, cell aggregates or organoids are selected from chondrocytes, human umbilical vein endothelial cells (HUVECs), myocytes, neuronal cells, kidney cells, stem cells or kidney organoids. In an embodiment the composition comprises between 0.25 and 30 w/v %, preferably between 1 and 10 w/v %, of the compound represented with formula (I), (IIa) or (IIb), for example 0.25, 0.5, 0.75, 1, 2, 3, 4, 5, 6, 7, 8, 9 or 10 w/v % of the compound represented with formula (I), (IIa) or (IIb).

In an embodiment the molar ratio of compound I to crosslinking agent is between 10:1 and 1:4, for example 10:1, 9:1, 8:1, 7:1, 6:1, 5:1, 4:1, 3:1, 2:1, 1:1, 1:2, 1:3 or 1:4.

In a further aspect the invention relates to the 3D printed structure obtained or obtainable by the method according to the invention.

It is envisioned that the hydrogels described herein can be used to embed cells. Of particular interest may be to use such hydrogel embedded cells as a model for a tissue or organ, or for drug screening, drug testing or drug validation. Therefore in a further aspect, the invention relates to the use of the hydrogel according to second aspect of the invention or the 3D printed structure according to the fourth aspect of the invention in one or more of:
drug screening,
drug testing,
drug validation,
culturing cells or organoids,
as a model organ or model tissue.

It is further envisioned that the hydrogels described herein are used as a transplant in a patient to replace or support a load bearing tissue such as, for example, cartilage, muscle or tendon. Therefore, in a further aspect the invention relates to the hydrogel according to second aspect of the invention or the 3D printed structure according to the fourth aspect of the invention for use in a surgical method, the method comprising implanting the hydrogel or the 3D printed structure in a subject in need thereof. The hydrogel may be a supramolecular hydrogel or a crosslinked hydrogel as described herein. The hydrogel for the purpose of transplanting may comprise cells, cell aggregates or organoids, however their presence is not mandatory and may depend on the intended application or implantation site.

### EXAMPLES

### Example 1-BTA Hydrogel

### Molecular Design and BTA Hydrogelator Synthesis:

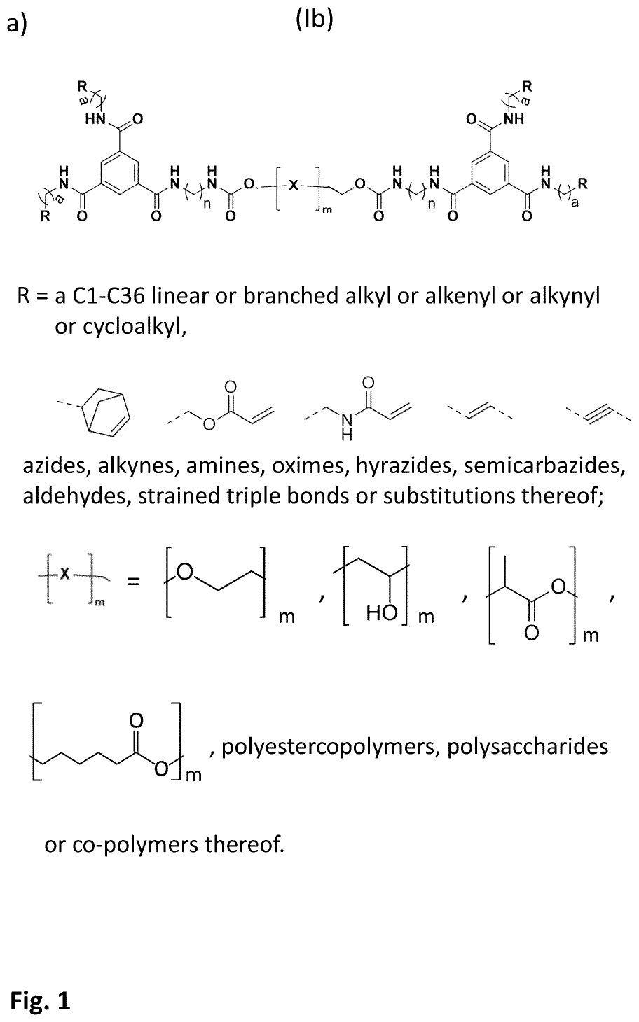

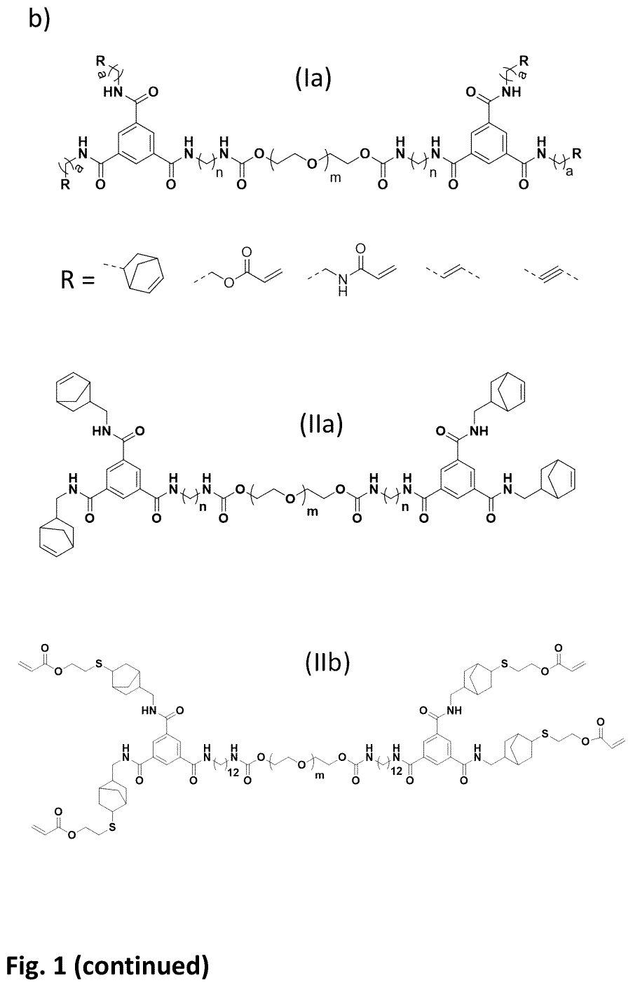

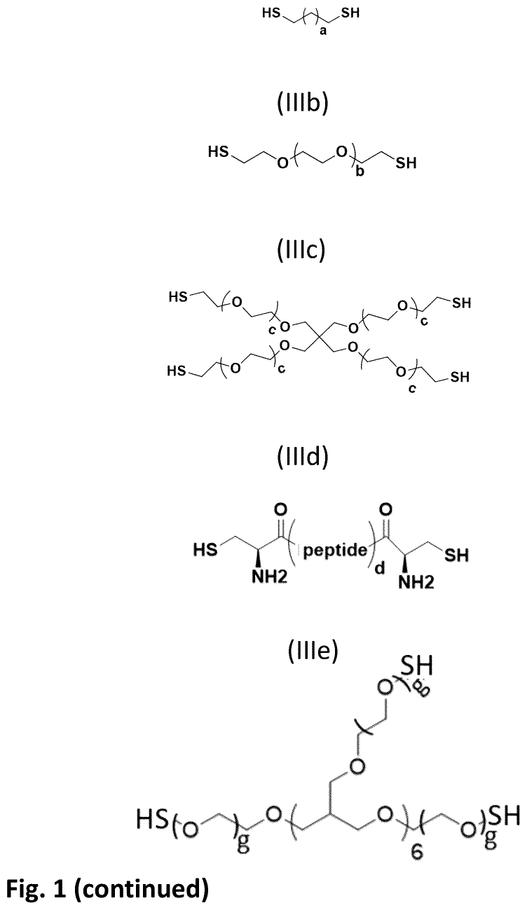

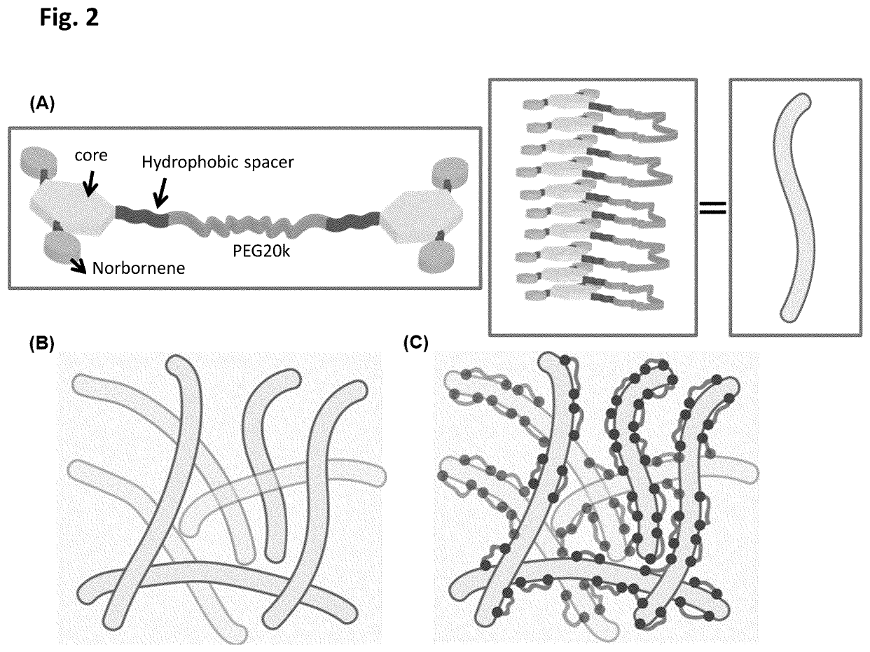

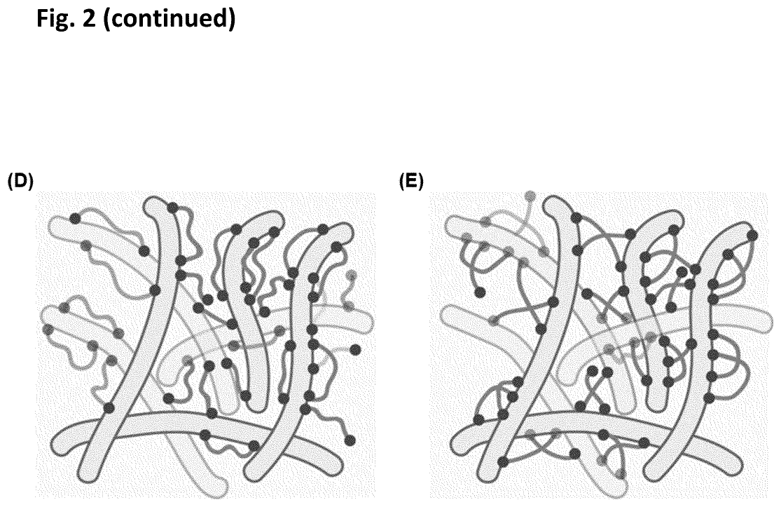

We designed BTA hydrogelator derivatives by introducing norbornene (Nb) orthogonal functionality on two outer ends of BTA (FIG. 1). We hypothesized that Nb would provide hydrophobic pocket for effective shielding from surrounding polar media since it is hydrophobic molecule and later can be employed for cross-linking supramolecular hydrogel by introducing interfiber and intrafiber cross-links using thiol cross-linkers (FIG. 1). Dodecane, twelve carbon atoms, was used as an internal aliphatic spacer. Dodecane was chosen based on previously published report that minimum eight carbon atoms are needed for effective shielding and stacking of BTA molecules⁴⁰. With previously reported desymetrization strategy, BTA synthon was synthesized by desymmetrizing a symmetrical 1,3,5-benzenetricarbonylpentafluoroester (BTE-F5Ph) molecule with 5-norborne 2-methylamine. Norbornene polymeric BTA hydrogelator are synthesized by connecting two BTA synthon molecules to PEG20K dodecane amine. We choose longer hydrophilic polymer PEG 20K to ensue good solubility in water. We successfully synthesized the hydrogelator on multi-gram scale with weight average molecular weight ˜23 kg/mol and D 1.1.

### Self-Assembly Studies in Dilute Solutions and in Gel Form.

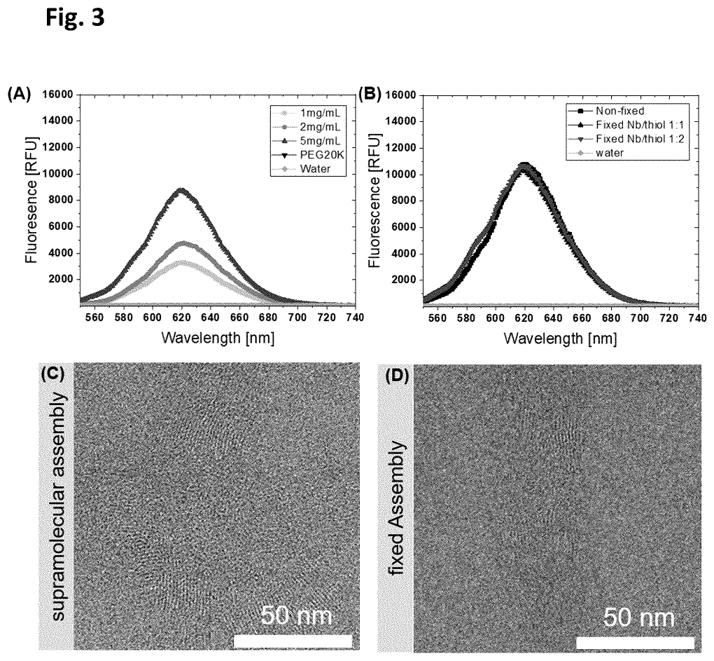

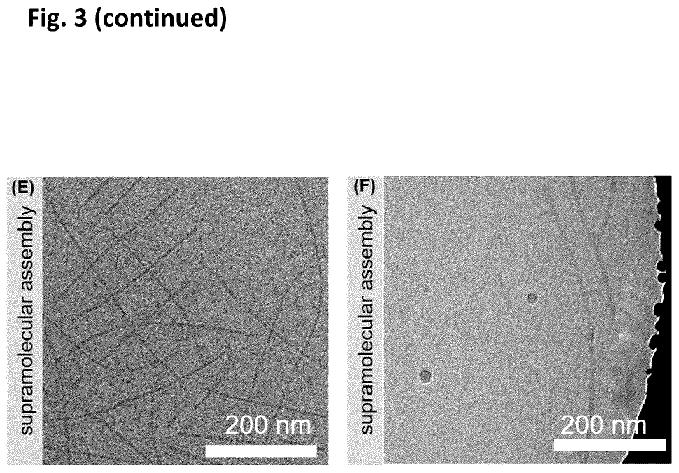

Having synthesized the polymer on multi-gram scale, we wanted to investigate the self-assembly of norbornene BTA in aqueous environment. For successful self-assembly, hydrophobics on BTA molecule should form a hydrophobic pocket in aqueous solution. This hydrophobic pocket can be investigated using Nile Red solvatochromic dye, which provides information about molecular environment of hydrophobic pocket formation. Nile Red shows high fluorescence signal in apolar solvents compared to water. A BTA solution in water showed an increase in fluorescence compared to water alone. Fluorescence intensity also increased with increasing concentration of the hydrogelator from 1 to 2 to 5 mg/ml. Mostly increase in fluorescence intensity was linear with increasing BTA concentration, which indicates increasing volume of hydrophobic pocket formation with increasing concentration of BTA hydrogelator. Maximum fluorescence intensity wavelength stayed constant for all tested concentrations (FIG. 3A). These results suggest a hydrophobic pocket formation with similar polarity within the BTA self-assembled structures.

After investigating hydrophobic pocket formation in supramolecular fibers, we wanted to investigate if fixing of hydrophobic pocket formation would change size or polarity of the hydrophobic pocket. BTA molecule has Nb on them and we employed thiol-ene chemistry for cross-linking BTA molecules. After cross-linking BTA molecules, we did not observe any increase or decrease in fluorescence signal and neither red nor blue shift was detected, which indicates that size and polarity of the hydrophobic pocket formations stays the same and was not affected by cross-linking of hydrophobic pocket and supramolecular structure (FIG. 3B).

Next, we investigate structure of self-assembled BTA molecules in dilute solution under cryo-TEM. We found large superstructures of BTA in 24 hours aged sample at 10 mg/ml of polymer BTA hydrogelator. Zoomed in areas shows that these structures consists of BTA nanofibers which are bundled together. Single nanofibers in bundles have diameter around 1.5 nm and are anisotropically parallel to each other in the bundle. A single bundle in superstructure has diameter around 15 nm (FIG. 3C&D). Fixing of the supramolecular structure does not change the structure and fibrous morphology was retained (FIG. 3D). We also investigated cryo-TEM structure of supramolecular hydrogel samples at 5 mg/ml and surprisingly could not observe the structure after 24 hours and aged the samples for a month and we found high aspect ratio long nanofibers with diameter around 5 nm and from a couple hundred nanometers to a few microns in length. Larger fibril aggregates of diameter around 15 nm diameter were also found and they are quite similar in diameter to fibril aggregates found in 10 mg/ml after 24 hours of aging.

### Gel Formation, Structural Analysis Via Cryo-SEM and Cross-Linking of Supramolecular Gel for Obtaining Stretchable and Tough Hydrogel

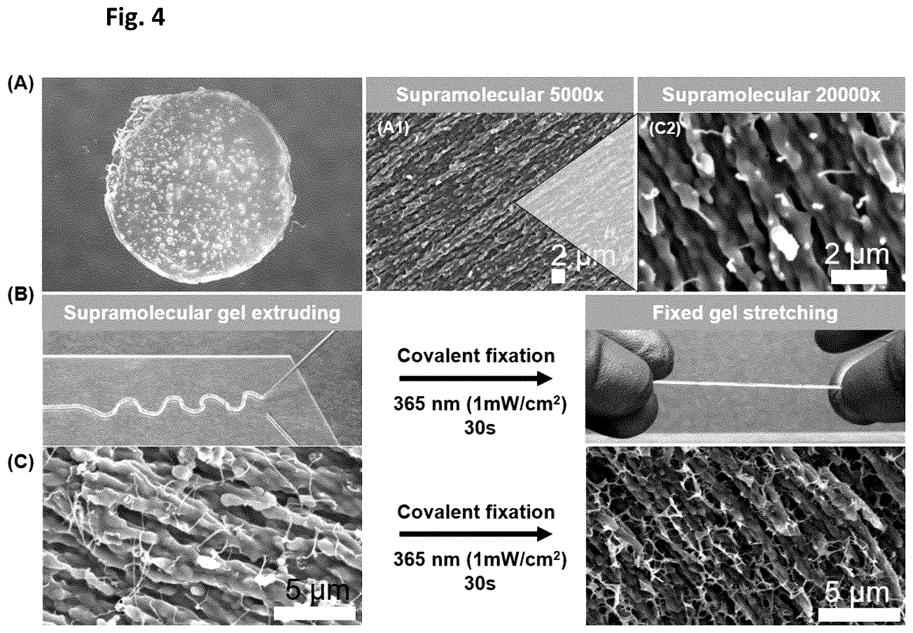

Next, ability of BTA to form hydrogel in water was assessed. Milli Q water was directly added into BTA powder and heated until polymer solution in water becomes turbid. BTA form clear nice gel upon cooling. In order to find suitable wt % of the polymer to form nice and stable gels, we tested different wt % of polymer i.e., 2.5%, 5%, 7.5%, and 10% (w/v) of polymer to form hydrogels in milli Q water. Hydrogelator form gels in water at all tested concentrations, while playing with hand we can feel that gels are soft for low wt % of the polymer and stiff for higher wt % of the polymer. Hydrogelator at 2.5% (w/v) forms a weak gel which is easy to deform and can turn into liquid upon even compressing gently. When cut the gels using spatula, all gels are cuttable and then upon placing them back together they offer excellent self-healing properties. Supramolecular gels also offer excellent extrudability, resulting in uniform continuous filament at 5% (w/v) and 10% (w/v) when extruded through standard 22 gauge (FIG. 4).

Next, we wanted to investigate if cross-linking of norbornene BTA supramolecular building blocks are possible. Cross-linking of BTA supramolecular building block will introduce interfiber and intrafiber cross-links. We will use the term cross-linking for interfiber and intrafiber cross-links in supramolecular hydrogel. For cross-linking supramoelcaulr building blocks in supramolecular hydrogel, a soaking strategy was developed. Premade norbornene BTA hydrogel at higher wt % was used and then di-thiol cross-linker and LAP solution was added and mixed. For uniform mixing, hydrogel was supramolecular hydrogel was chopped into pieces and placed at room temperature from minimum 30 mins until 3 hours for soaking of cross-linker and LAP solution. Soaking step was introduced for uniform permeation of LAP and di-thiol cross-linker in the hydrogelator. After soaking, hydrogels are exposed to UV-light at 365 nm wavelength for 30 seconds at 1 mw/cm². Interestingly, all tested formulations resulted in strong hydrogel compared to before cross-linking by exposing to UV-light.

Next, we were tempted to visualized hydrogel under scanning electron microscope for visualizing structure of supramolecular hydrogels and if there are any structural changes after cross-linking (FIG. 4). We used transmission electron microscope (cryo-TEM) for investigating hydrogel structure. Scanning electron microscopy images showed bundle of fibers for supramolecular hydrogel. Supramolecular hydrogel exhibit nice extrudability and we investigated morphology of extruded fiber before and after cross-linking. SEM micrographs revealed that extruded gel showed bundle of fibers before and after cross-linking and no change in structure was observed after extruding. Extruded hydrogel before and after cross-linking showed anisotropic, highly aligned bundle of fibers before and after cross-linking (FIG. 4).

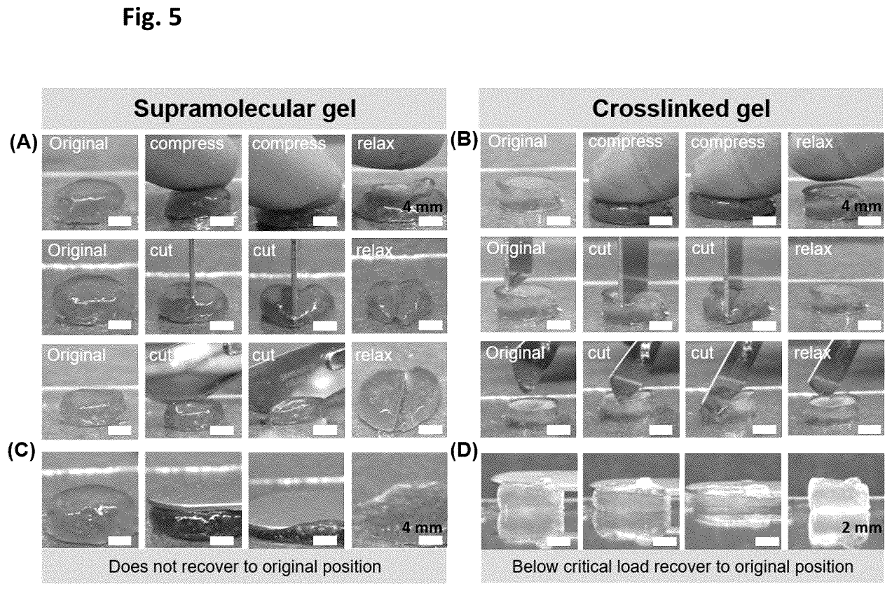

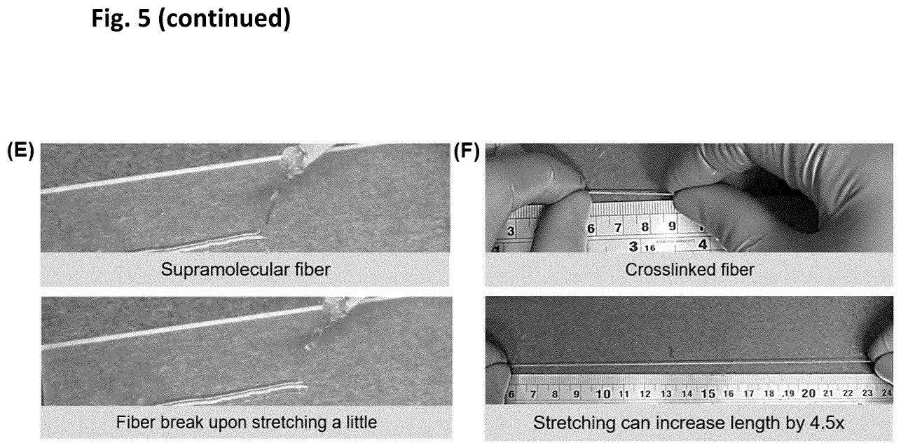

Having established that cross-linking of supramolecular hydrogel does not alter anisotropically aligned fibril structure of the hydrogels, we wanted to understand macroscopic properties of hydrogel after cross-linking. We directly compared supramolecular hydrogel with cross-linked hydrogel. Hydrogels were cross-linked with short di-thiol cross-linker. Supramolecular and cross-linked hydrogel were both compressible and cross-linked hydrogel showed immediate recovery while supramolecular hydrogel did not show any noticeable recovery. Supramolecular gel can be cut using spatula while cross-linked hydrogel offer strong resistance and even sharp blade did not cause much damage to the cross-linked hydrogel (FIG. 5A&B).

Supramolecular gel does not return to original position after compressing and cross-linked hydrogel can return to original position within seconds (FIG. 5C,D,E&F). Cross-linked hydrogel bears significant load and crack only started to appear under extreme deformation along the edge of the spatula. Interestingly, crack did not propagate when the load through the network and had to even press stronger for crack to progress into hydrogel. This perhaps can be because of fibril nature of hydrogel and fibers can stop propagation of the crack. Supramolecular gel can flow under gravity and close small crack and fixed gel do not flow but it can self-heal and only break when pulled with significant force. Extruded supramolecular fiber is weak and break when stretched and cross-linked fiber can be stretched above to 4.5 times its original length (FIG. 5G&H).

### Tunable Mechanical Properties by Changing Polymer Weight Ratio, Cross-Linker Mole Equivalents and Cross-Linker Length and Functionality

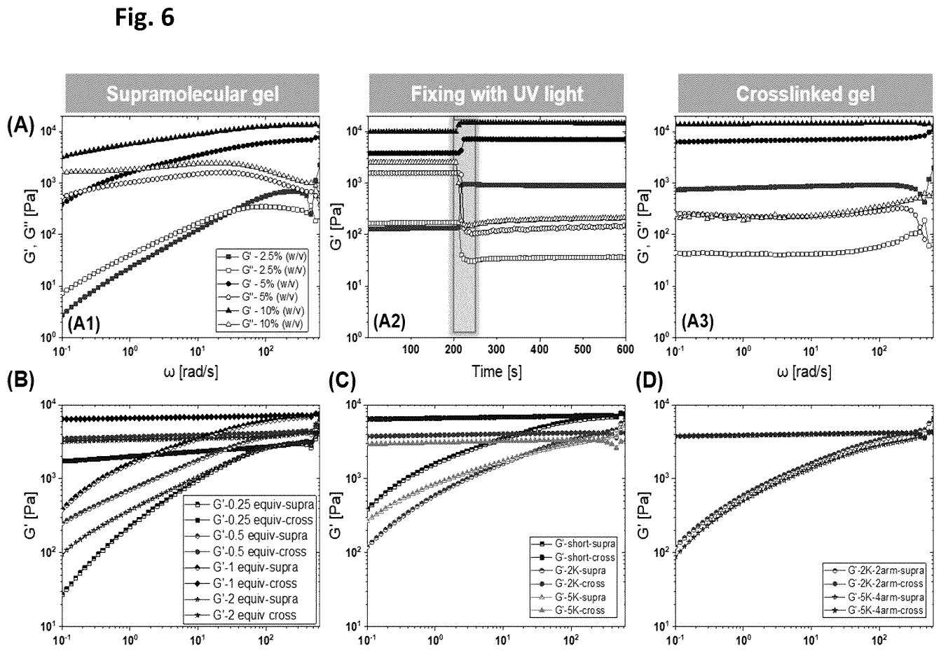

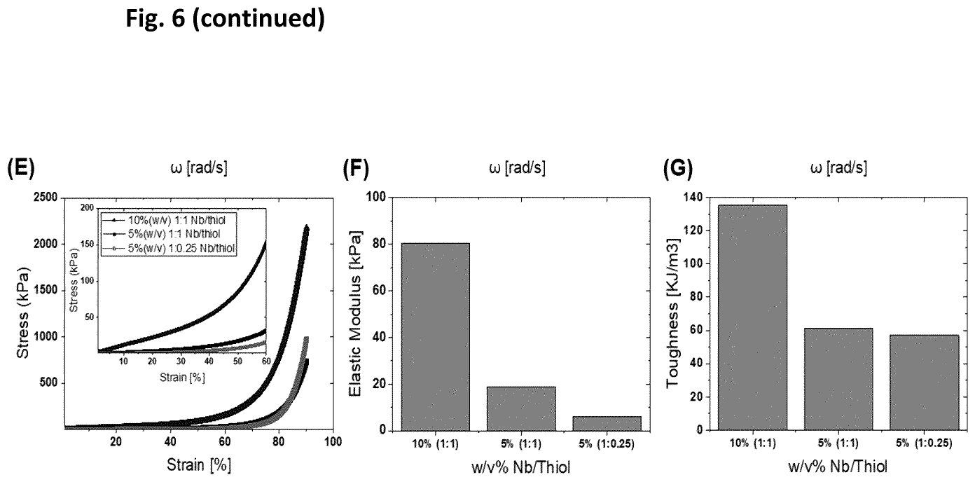

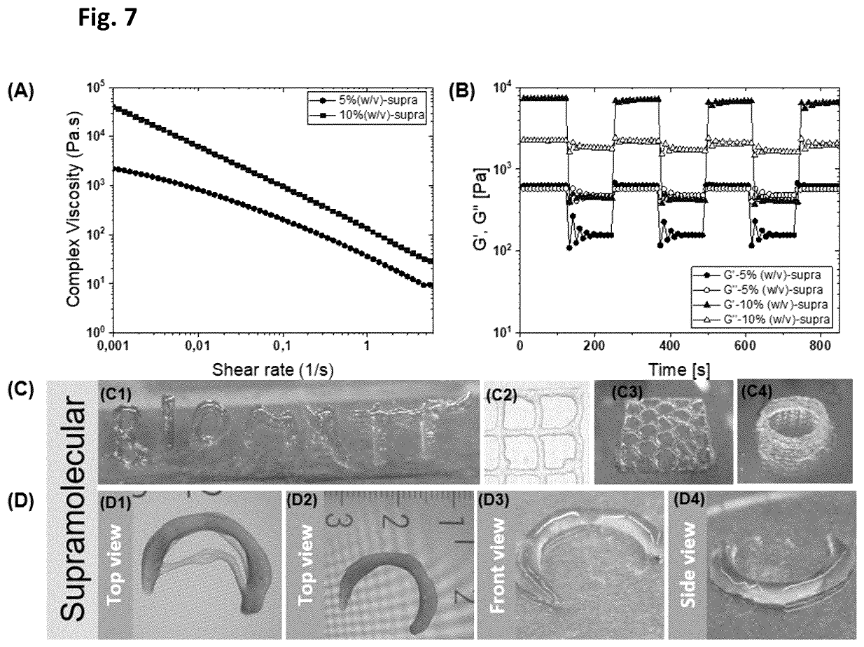

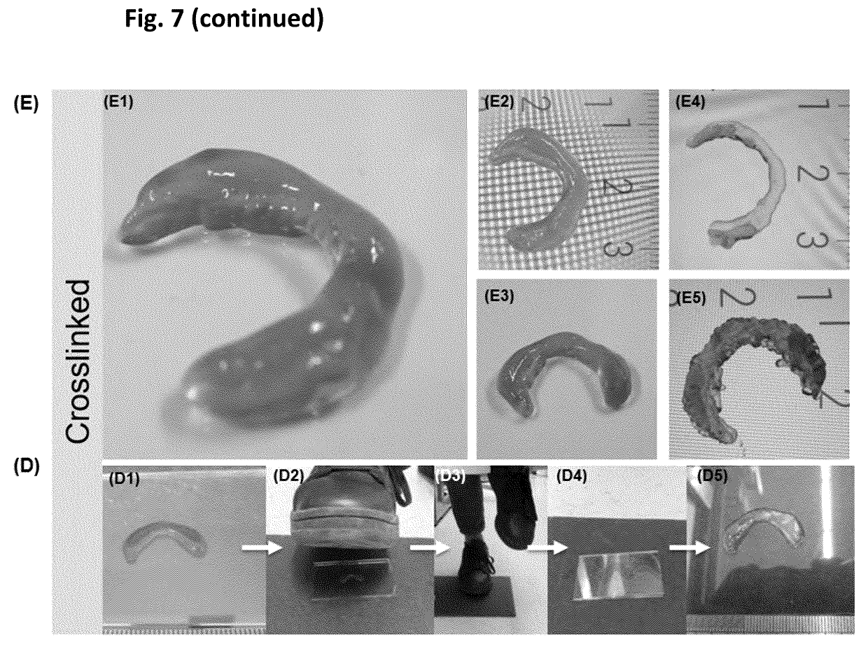

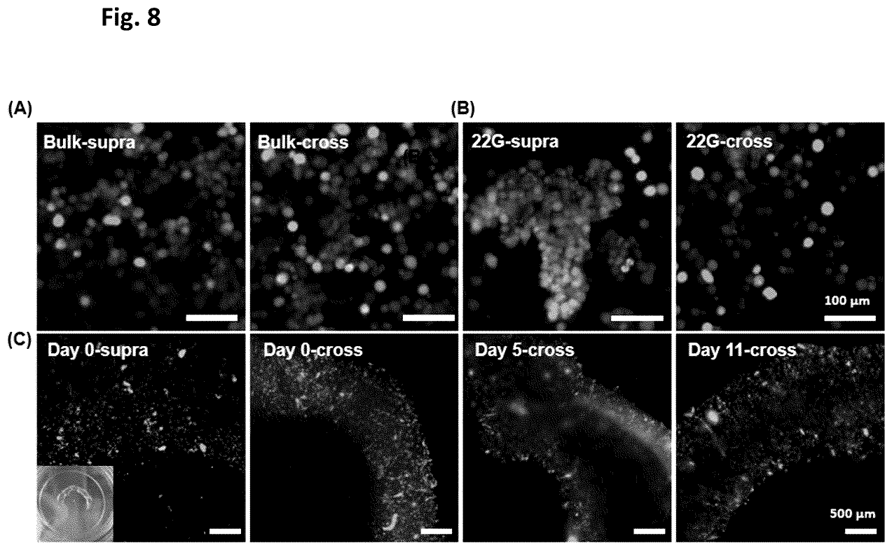

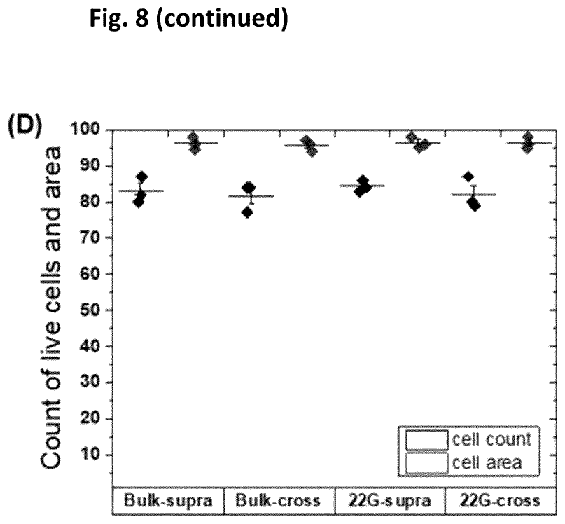

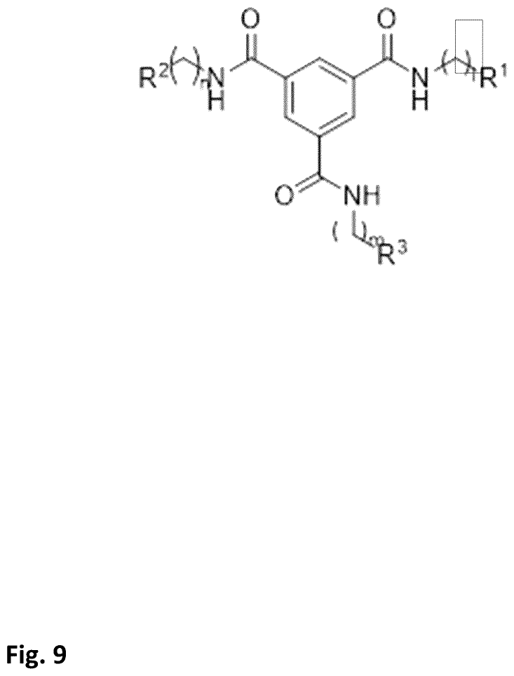

Mechanical properties of hydrogel can be tuned with changing polymer concentration. To verify our hypothesis, we determine mechanical properties of hydrogel by changing wt % of the polymer (FIG. 6). Storage moduli of hydrogel increased with increasing concentration of polymer which perhaps can be attributed to more physical interaction between fibers at higher % (w/v) of the polymer. Not only storage moduli but also viscoelasticity of hydrogel can be tuned by changing concentration of BTA hydrogelator with more viscoelastic character at low % (w/v) of the polymer. BTA hydrogelator showed a crossover point at 34 rad/s for 2.5% (w/v) and decreases to 0.25 and 0.01 rad/s for 5% and 10% (w/v), which indicates decrease in viscoelastic character with increasing hydrogelator concentration. Viscoelastic character shows dynamic and reversible nature of supramolecular interactions within BTA hydrogels and that hydrogels are viscoelastic fluids.

Next, we wanted to investigate if supramolecular gels can be cross-linked using thiol-ene orthogonal chemistry. Short thiol cross-linker were mixed with all formulation in 1:1 ratio of norbornene to thiol. LAP photoinitiator was used and gels were exposed to UV light at 10 mW/cm² on rheometer at 1% strain and 1 rad/s angular frequency. All formulations cross-linked quickly under just few second, which shows that thiol-ene chemistry is highly efficient (FIG. 6-A2). Cross-linking of supramolecular gels was performed at 10 rad/s and clearly can be seen that 2.5% (w/v) was predominantly liquid and turns into solid after cross-linking. Supramolecular hydrogel with 5% and 10% (w/v) were already dominantly solid and upon cross-linking showed an increase in storage moduli (FIG. 6-A2). Storage moduli and loss moduli of cross-linked hydrogel stayed constant in angular frequency sweep (FIG. 6-A3) indicating that cross-linked hydrogel are dominantly viscoelastic solids in contrast to viscoelastic fluids before cross-linking. Interestingly, storage moduli of the cross-linked hydrogel is similar to equilibrium storage moduli of supramolecular hydrogel which indicates that cross-linking of hydrogel did not change hydrogel structure.

After establishing that hydrogel can be cross-linked and mechanical properties can be tuned as function of polymer concentration, we hypothesized that storage moduli can also be tuned by varying the degree of cross-linking at constant polymer concentration. We varied degree of cross-linking by varying the number of mole equivalents of short di-thiol cross-linker. To this end, a series of hydrogel with 0.25, 0.5, 1, and 2 mole equivalent of short di-thiol cross-linker were prepared. Short di-thiol cross-linker was mixed with hydrogels and angular frequency sweep was run to investigate influence of presence of short di-thiol cross-linker on viscoelastic properties of BTA hydrogel. The storage modulus increased with increasing cross-linker mole equivalents and was highest when norbornene to thiol equivalent ratio was 1:1. Storage modulus was 4000 Pa for 0.25 equivalents and increased to around 6000 Pa for 0.5 equivalents and to ˜8000 Pa for 1 equivalent. Storage modulus decreased to 5000 Pa for 2 mole equivalents suggesting decrease in degree of cross-linking perhaps owing to formation of dangling ends and loops which would not effectively contribute to storage modulus. We observed that presence of thiol cross-linker already influenced the equilibrium storage moduli in supramolecular hydrogels and interestingly cross-linked hydrogel showed similar equilibrium storage modulus to supramolecular hydrogel equilibrium storage modulus.

Next, we hypothesize that molecular length of cross-linker can affect mechanical properties since shorter molecular cross-linker length would preferably cross-link BTA molecule in the same fiber (intrafiber) and less interfiber (between fibers) cross-links since it is only eight atoms long and longer cross-linker can cross-link BTA molecule at larger distances which would have larger possibility to make interfiber cross-links compared to intrafiber. Total number of functional groups were kept same and in ideal case that would result in equal number of cross-links and only variable was length of cross-link. PEG short di-thiol cross-linker, PEG2K di-thiol, PEG5K di-thiol and PEG20K di-thiol cross-linker were used for cross-linking. Hydrogels with cross-linker mixed and before cross-linking showed typical viscoelastic spectrum (crossover point between G′ and G″) besides PEG20K di-thiol cross-linker. However presence of cross-linker does affect mechanical properties of hydrogels and with increasing cross-linker length a small gradual decrease in storage moduli was observed from roughly around 8000 Pa for short cross-linker to 4000 Pa for 20K PEG cross-linker. Next, we fixed the gels using UV light and all the cross-linker cross-linked the gels instantaneously. All fixed hydrogels showed a plateaued G′ and G″ over almost 4 orders of magnitude angular frequency demonstrating dominant solid-like behaviour. Interestingly, storage modulus for all cross-linked hydrogel is similar to equilibrium storage modulus of supramolecular hydrogel before cross-linking. This indicates that even cross-linker with larger molecular weight did not alter the structure of supramolecular hydrogel during cross-linking.

Network topology of cross-linker can also affect mechanical properties owing to step growth nature of norbornene thiol-ene cross-linking. We compared 5K-PEG-4arm versus 2K-PEG-2arm for investigating mechanical properties. When comparing supramolecular hydrogels with cross-linker present both hydrogels were viscoelastic and showed a crossover point and at similar angular frequency and also showed very similar equilibrium storage modulus. When cross-linking, 2 arm cross-linker showed fast cross-linking compared to 4-arm. Cross-linked hydrogels for 5K-PEG-4arm and 2K-PEG-arm showed plateau storage modulus of around 4500 Pa independent of frequency. Total number of end functional groups are same for both cross-linker and theoretically speaking both would form the same number of new bonds. However, 5k-PEG-4arm cross-linker would have one extra cross-link since two 2arm PEG chains are covalently attached for the 4-arm PEG cross-linker. Taking into consideration theoretical number of bonds, 5K-PEG-4arm should show higher storage modulus compared to 2K-PEG-2arm. Apparently it seems like 5K-PEG-4arm formed less number of new bonds compared to 2k-PEG-2arm which perhaps can be attributed to less availability of either thiol or norbornene or not being able to find each other in the vicinity of each other for 5K-PEG-4arm.

Having established that stiffness can be tuned in these hydrogels, we next explored whether covalent fixation of supramolecular hydrogel can result in enhanced elastic modulus, strain at break and toughness of the hydrogel. We moved to investigate these gel further with compression and tensile testing machine since rheometer does not provide accurate information about strain at break and gels started to slip at a few hundred percent strain. Typical compression stress-strain curve is shown in the FIG. 6E and 10% (w/v) gel shown significantly higher stress at 90% strain (2.5 MPa) compared to 5% (w/v) gel with 1:1 and 1:0.25 molar cross-linker concentration. None of tested hydrogel fracture under 90% compression strain and once the stress was removed, 10% (w/v, 1:1) recover quickly to its original dimensions, 5% (w/v, 1:1) recovered between 5-10 minutes and 5% (w/v, 1:0.25) would take roughly 30 minutes. We also observed that 5% (w/v, 1:0.25) was sticky the most sticky and would hold compression plates together after running the compression test. Elastic modulus was around 80, 20, and 5 kPa for 10% (w/v, 1:1), 5% (w/v, 1:1) and 5% (w/v, 1:0.25) which is in the range for soft to hard tissues.

We compressed the hydrogel to 90% strain and stresses ranged from 500 kPa (0.5 MPa) to 2500 kPa (2.5 MPa) shown in FIG. 6F. These level of stresses are in the range of soft biological tissues such as cartilage⁴⁴ and human Annulus Fibrosus⁴⁵ in their typical stress-strain curve. We also calculated toughness of the hydrogel and highest toughness around 140 KJ/m³ was found for 10% (w/v, 1:1) and 60 KJ/m³ for 5% (w/v, 1:1) and 5% (1:0.25). Interestingly, 5% (w/v) showed similar toughness even at two different concentration of cross-linker.

Load bearing soft collagenous tissues usually exhibits a bimodal stress-strain response: a compliant response at low strains and stiff response at large strain 46. Our hydrogel also showed compliant response at low strain and then stiff response at higher strain where stress increased non-linearly with applied strain. Based on the structure of BTA hydrogelator and the stress responses observed, we believe that first non-covalent interactions between BTA building blocks continue to deform and dissipate stress at low strains and then stiffening response started with covalent bonds deformation and continue to become dominant with increasing strain as stress rapidly increased. It is interesting to note that critical value of strain at which stress-strain showed divergence from linear relationship or stress increases rapidly is different for different BTA formulations. Rapid stiffening behaviour started around 30-40% for 10% (w/v) compared to 50-60% strain for 5% (w/v) (FIG. 6E). This perhaps can be attributed to higher degree of cross-linking for 10% (w/v) gels compared to 5% (w/v) and early onset of covalent bond rupture.

### Rapid Shear-Thinning, Self-Healing Properties Impart Injectability and 3D Printability:

After tuning mechanical properties, we aimed to investigate hydrogel for injectability and 3D printing applications. A shear-thinning hydrogel shows decreases in viscosity upon application of strain or shear rate and is considered an ideal material for 3D printing applications. Self-healing is another property required for hydrogels to quickly reform the network and maintain printed structure shape fidelity. Shear-thinning and self-healing property of BTA hydrogels were investigated at 2.5%, 5% and 10% using shear rheology. All BTA formulations show shear-thinning behaviour. At shear rate of 6 (1/s) BTA at 10% (w/v) and at 5% (w/v) showed complex viscosity around 30 Pa·s and 8 Pa·s. We also ran continuous flow sweep for determining shear-thinning behaviour and BTA at 10% (w/v) and at 5% (w/v) showed viscosity of 13 Pa·s and 25 Pa·s at shear rate of 10 (1/s). Shear-thinning experiment confirms rapid shear-thinning behaviour of hydrogel at considerably low shear rate. After shear-thinning, we investigated self-healing ability of hydrogel to recover quickly to their original storage moduli. Hydrogels also showed self-healing behaviour since hydrogels goes to liquid (G″>G′) in shear rupture cycle at higher strain (400%) and showed quick network recover to original properties at low strain (1%) under just a few seconds. BTA hydrogel show rapid shear-thinning and self-healing properties and can be attributed to quick on and off rate of hydrogen bonding interactions in BTA hydrogel.

After establishing that supramolecular hydrogels offer shear-thinning and self-healing properties, next we demonstrated capability of supramolecular hydrogel to be extruded and 3D printing into different shapes with good shape fidelity post extruding. We explored different wt % of BTA hydrogels for optimization of shape fidelity. We used 5% and 7.5% for printing a filament and multilayers for determining uniformity of filament and shape fidelity by printing multiple layers. Both formulations showed above 90% filament uniformity and with good shape fidelity post printing. Higher concentration of the hydrogel 7.5% (w/v) took 200 kPa pressure to extrude compared to 90 kPa pressure for 5% (w/v). We used 5% (w/v) gel for all next 3D printing experiment since we were interested to investigate hydrogel for bioprinting applications, which can allow to bio-print encapsulated cells safely without cell membrane damage below 120 kPa.

We extruded different logos BIOMATT, MERLN, CTR and 3D printed grid-like and hexane-like structure at 5% (w/v) using 22 gauge. All of the structures were printed with uniform filaments and good retain their shapes post printing without secondary cross-linking. Next we investigated ability of hydrogelator for printing a solid-tissue like structure. We know from mechanical properties that hydrogel exhibit excellent compression properties and cartilage was clear choice to print. We designed a 3D model of cartilage and printed hollow and solid cartilage model using supramolecular hydrogel. Hydrogel can be printed nicely and transition from thick to thin edges can me made smoothly and hydrogel stayed stable post printing. Important to note is that hydrogel is supramolecular and have not been cross-linked. 3D printing into complex shapes and maintaining good shape fidelity is possible owing to excellent shear-thinning and self-healing properties of hydrogel. Also this highlight the importance of supramolecular interactions and self-assembly as important designing parameter for developing hydrogels which can maintain structure and shape post-printing.

Next, we printed again printed cartilage model and this time we wanted to investigate if we can cross-link the hydrogel while we are printing for maintaining small resolution features on cartilage edges. We mixed short di-thiol cross-linker and LAP photoinitiator and cross-linked the hydrogel after printing each layer or after printing the whole construct. 3D printed cartilage model retained very fine details of 3D cartilage model. The printed and cross-linked hydrogels are tough and can undergo extreme deformations. To demonstrate toughness, we placed the hydrogel in between two glass plates and a student of 60 kg weight stood for 30 seconds and repeated the process thrice. They hydrogel did not break and absorb all the stress. After unloading, we observed that hydrogel recovered more than 95% of its original thickness within 5 minutes.

Tissues interfaces usually possess gradient of mechanics in space for example as in cartilage tissue stiffness increases going from superficial soft zone to calcified deep zone⁴⁷. Cartilage zonal gradient structure can be printed using layer by layer approach and stiffness gradient can be created by printing inks layers with different stiffness. Moving forward, we show proof of concept by using ink formulations with different thiol cross-linker for changing stiffness. Using inks with 0.5 and 1 mole equivalent of short thiol, multi-material printing was utilized to deposit inks at different sites on cartilage model to show that inks can be deposited spatially with desired mechanical properties. Food grade color green and red was added for clear visualization. Importantly, the same hydrogel material with different thiol cross-linker concentration can be printed and range of mechanical properties from soft to hard can be accessed. Excellent self-healing properties of material allow materials to heal after printing and then cross-linked as a single solid part without any discontinuities or cracks. Self-healing properties of BTA hydrogel owing to dynamic hydrogen interactions and ability to tune mechanical properties in a single system which offer biomimetic fibril structure highlights the importance of rational design of hydrogelator for 3D printing.

Unlike traditional inks, which are viscous liquids, supramolecular hydrogel owing to transient and dynamic interactions offer shear-thinning and rapid self-healing properties essentially required for 3D printing with good shape fidelity. Importantly, supramolecular inks provide freedom to design inks at molecular scale, control structure at nanoscale and develop bioinks with structure-property relationship. Looking ahead supramolecular inks offer novel way for designing inks by engineering supramolecular interactions and create structures at nanoscale which has not been possible using traditional bioinks. Utilizing supramolecular polymers with noncovalent interactions, we can take advantage of specificity of supramolecular interactions and modular inks with multifunctionalities can be designed.

Cell Viability in Bulk Gels, Post Injection and after Bioprinting:

PEG hydrogels are known to be biocompatible and have been extensively used in biomedical applications. BTA hydrogels have PEG as polymer backbone and BTA as building block which undergo hydrogen bonding interaction resulting in hydrogels. We hypothesize that BTA hydrogel can be used to encapsulate cells and cell viability within gels can be maintained. ATDC5 chondrocytes were encapsulated within BTA hydrogels using rapid self-healing ability of hydrogel. After encapsulation hydrogels were stained calcein (green=live cell) and ethidium homodimer (red=dead cell) for investigating cell viability. Cells were stained after 2 and 24 hours of encapsulation. Qualitatively can be seen that the majority of cells are live after 2 hours. We counted the number of live and dead cells and greater than 80% of cells were alive. Since cells tend to clump together and aggregate of cells would be counted as a whole single cell in image J analysis, which could lead to underestimation of the total number of a live cell. Therefore, we also calculated the area of live and dead cells. Cell area calculations showed that greater than 90% of cell area belongs to live cells. After determining cytocomaptibility in supramolecular hydrogel, we investigated cell viability in cross-linked hydrogel, hydrogel was cross-linked at 1 mW/cm² for 30 seconds. Cross-linked hydrogel also showed similar cell viability (>80%) to supramolecular hydrogel demonstrating that hydrogel cross-linked successfully in presence of cells and that exposure to UV did not affect cell viability.

Hydrogels offer rapid shear-thinning and self-healing properties, which are desirable for localized delivery of cells via injection without compromising cell membrane and cell efficacy. Many studies have shown that cell viability is reduced when cells are passed through syringe needle flow and that shear-thinning self-healing hydrogel provide protection from shear stresses during flow through the syringe. We investigated BTA hydrogel potential as injectable cell carrier. ATDC5 encapsulated within hydrogels were extruded through 22 gauge and investigated for cell viability before and after cross-linking by exposure to UV light at 1 mW/cm². Large percentage of cells (>80% cell count and >90% cell area) were alive in extruded supramolecular gels and also in extruded cross-linked gel demonstrating that BTA hydrogel provide protection to cell membrane damage when extruding through 22 gauge needle and also post-extrusion cross-linking did not decrease cell viability. BTA hydrogel is interesting for clinical applications since hydrogel may require to be in injectable state for hours before cross-linking; however, most of the hydrogel are rapidly cross-linking after injection. BTA gel is injectable even after 3 hours, can adopt different mold shapes and can be cross-linked once hydrogel take the shape of surgical defect. BTA hydrogel can be suitable as an injectable material and may overcome the flaw that current injectable hydrogel material has.

After demonstrating that BTA hydrogel rapid shear-thinning and self-healing provides protection while passing through syringe, we investigated bioprinting of complex life-like structure. We bioprinted cartilage model and ATDC5 chondrocytes were used since they are widely studied cell type for cartilage tissue engineering and cartilage-like matrix production. We encapsulated ATDC5 chondrocytes using the self-healing ability of hydrogels and bioprinted into cartilage shape and cell viability was determined by staining with live-dead assay over day 1, day 5 and day 11. Cell stayed viable until day 11 and no decrease in viability in bioprinted hydrogel were observed.

### Example 2-Synthesis

### Activated 1,3,5-Benzene Tricarbonyl Triester (BTE) Library Synthesis:

In attempting to create BTAs of lower symmetry, the most convenient approach would be desymmetrization of a commercial starting material. 1,3,5-benzene tricarbonyl trichloride (BTCI) would be an ideal candidate, though the acid chloride functionality would not be envisioned to be stable to purification. Nevertheless, we attempted to react BTCI with one equivalent of hexylamine to investigate its suitability as a synthon. In the absence of side products, the reaction would result in three product molecules (monosubstituted, disubstituted and trisubstituted) and leftover starting material (upon workup it is expected that the acid chlorides would hydrolyze into acids). Upon ¹H NMR analysis of the reaction mixture, numerous peaks appeared in the aromatic region and we could not easily obtain useful information on the constituency of the complex mixture formed during the reaction. This result was further supported by a long continuous streak on TLC without distinct spots in the reaction mixture. The reaction was run at different temperatures (20, 4, and −75° C.) to see if the temperature could clean up the product profile; however, all attempts resulted in similar peaks on ¹H NMR spectrum. Testing this reaction confirmed our suspicion that desymmetrization via an activated ester approach was the best way forward.

In order to find an alternative to BTCI that can facilitate desymmetrization via aminolysis, the formation of activated esters including aromatic carboxylic esters and thioesters are appealing approaches and have remained unexplored to create BTA derivatives. Aromatic carboxylate esters were determined to be a better choice since they are less susceptible to hydrolysis and more stable compared to thioesters. Commonly employed phenols and N-hydroxysuccinimide, which are commercially available, were chosen for creating a library of activated 1,3,5-benzene tricarbonyl triester derivatives (BTEs) (1-4, Scheme 1). Phenols with differences in pKa value were chosen with pka of 8.36, 7.15, 5.4 and 6.0 for 3-nitrophenol (3NO₂Ph), 4-nitrophenol (4NO₂Ph), 2,3,4,5,6-pentafluorophenol (F₅Ph), and N-hydroxy succinimide, respectively.

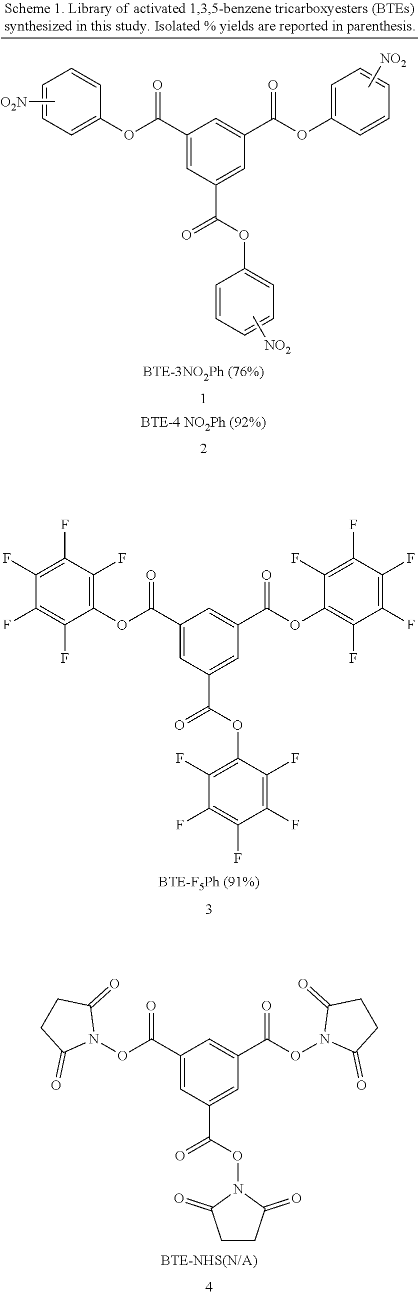

The aim of creating the BTE library was to investigate the ease of synthesis, stability and desymmetrization potential via controlled aminolysis of BTEs. We started our investigation with the synthesis of nitrophenol activated esters from BTCI (Scheme 1). Both the 3-NO₂Ph and 4-NO₂Ph show limited solubility in many solvents, yet tetrahydrofuran (THF) was found as a suitable solvent faciltating reaction completion in <6 h. After optimization of purification 1 could be obtained in good yield (76%), and 2 could be obtained in excellent yield (92%) by recrystallization. Next, the library was expanded and we synthesized 3 (Scheme 1) by coupling F₅Ph to BTCI. Due to the high solutiblity fo F₅Ph, this reaction could be easily run in DCM in under 4 hours. The reaction was clean, and TLC showed only two spots (Rf ˜0 and 0.9). The reaction mixture could be easily passed through a filter to remove the DIPEA salt and then through a bed of silica to yield 3 in 91% yield.

Attempting the synthesis of 4 proved problematic. NHS offered limited solubility, aside from THF and DMF. Running the reaction in DMF produce only non-symmetrical derivatives (based on ¹H NMR spectrum), while running the reaction in THF produced the symmetrical target compound 4. While TLC analysis of the reaction showed two spots, 4 was not able to be fully isolated from free NHS under numerous mobile and solid phases. Furthermore, pure 4 was not obtained via crystallization, and during numerous work-up attempts the ¹H NMR evolved extra peaks suggestive of degradation. Importantly, our experiments show that production and isolation of 4 is not straightforward and appears to be very sensitive to degradation during handling.

### Desymetrization of Activated BTE Synthons

After a mostly successful BTE library synthesis, we moved to investigate the desymmetrization potential of the symmetrical BTEs. Via stoichiometric control, we aimed to maximize the % yield of monosubstituted and disubstituted derivatives. For reference, previous statistical simulations showed a maximum of 37% monosubstituted derivative using one equivalent of the nucleophile²⁷ for a triply reactive system. Using hexylamine as a model nucleophile, we set out to create monosubstituted derivative (using one equivalent (per BTE synthon). DMF was found to be the best solvent for 1 (although not fully soluble), and during the reaction dissolution occurred.

In the absence of any side products, desymmetrization of molecule 1 (Scheme 2) would result in five molecules in the reaction mixture (monosubstituted, disubstituted, trisubstituted, free phenol, and remaining 1). TLC of the reaction mixture of 1 showed three spots suggesting that some products were not formed or only formed in small amounts.

Reaction mixtures resulting from the substitution of molecule 1 were partially soluble in DCM and the products could be isolated via column chromatography, though 7 and the trisubsituted BTA could not be fully resloved. ¹H NMR analysis (chemical shift, peak splitting pattern and integration analysis of peaks) was used to identify the compounds. Most importantly, this separation allowed us to identify the complex aromatic peak splitting found in the reaction mixture and assign peak patterns to the mono-, di-, and tri-substituted derivatives (doublet and triplet at 8.95 and 8.92 ppm for 5, doublet and triplet at 8.70 and 8.65 ppm for 7, and singlet at 8.34 ppm for the trisubstituted BTA).

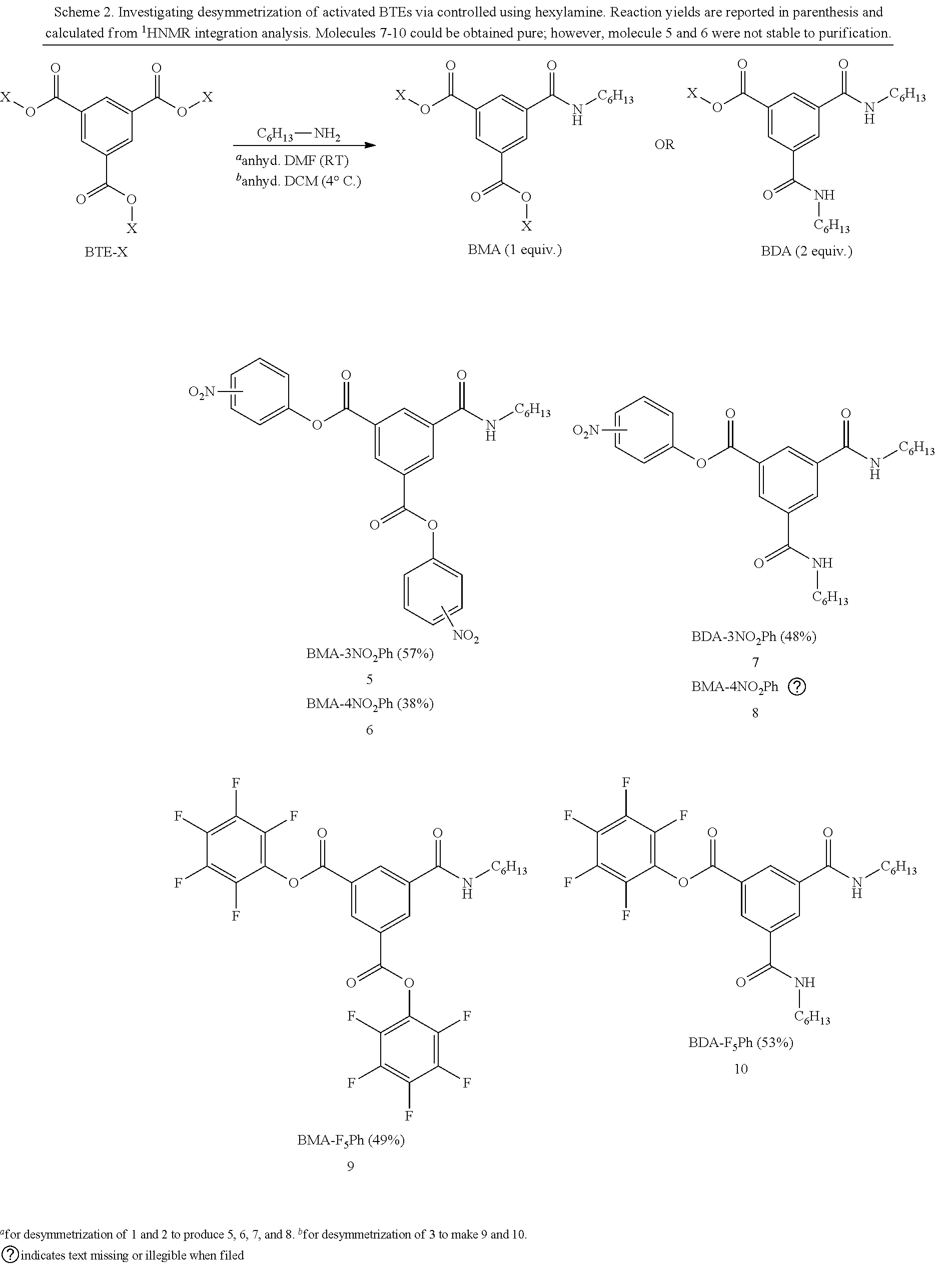

With the knowledge of proton chemical shift and peak splitting pattern of products, we were able to analyse the crude reaction mixtures and quickly determine the relative amount of the products in the reaction mixture. Molecule 1 produced a 57% yield of 5 (monosubstituted) when treated with 1.0 equiv nucleophile. The desymmetrization of 2 behaved similarly, and after separation and characterization the crude reaction mixture produced a 38% yield of 6 (monosubstituted derivative). Interestingly, 1 produced the monosubstituted product significantly higher than statistically calculated, while 2 produced almost equal to statistcially predicted. When the same reaction was run for two equivalents of hexylamine, 1 produced 7 (disubstituted) in 48% yield and 2 produced 8 in 54% yield. Interestingly both produced disubstituted derivative roughly twice than statistically predicted, which is 28% using two equivalent of nucleophile 27. With promising results, we then turned to desymmetrization of the penta-fluorophenol synthon, 3. After running the reaction with 1 equiv of hexylame in in DCM, the crude reaction mixture showed five spots on TLC, which were isolatable via column chromatography. After ¹H NMR analysis, we were able to determine the ratio of substituted derivatives in the crude reaction mixture (a similar pattern of doublet/triplets and singlets was observed as in the desymmetrization of 1). Peak integration from the ¹H NMR spectrum of the crude reaction mixtures showed that 3 produced 49% of 9 and 53% of 10 using one and two equivalents of hexylamine, respectively. Both desymmetrization reactions yielded higher than statisctical yields and the products were able to be readily isolated via column chromatography. When developing a reactive synthon for desymmetrization, stability and scalability are also important factors to consider. Both 1 and 2 were stable in a desiccator when stored for a year; however, upon handling in the lab over 2-3 months both molecules started to hydrolyze (¹H NMR). We did observe that 1 was more stable than 2. In comparison, 3 showed excellent stability in the lab; it was found to be stable for more than two years, even after open handling in a humid environment (the Netherlands). Purification of 1 and 2 required a large volume of solvents owing to limited solubility (100s of mL for 10s of mg), while 3 offered a short one-step work up with good solubility.

Due to its ease of synthesis, stability, desymmetrization, and purification, 3 was determined to be the best candidate to work with moving forward. We found 3 was stable over years under an inert atmosphere, stable in the humid environment of the lab, purified in a short one-step workup, easily scaled to gram scale, showed good solubility in low boiling point solvent, and showed simple and straightforward NMR analysis. Furthermore, the stability of the F₅Ph esters on 3 also offers easy separation of desymmetrized intermediates using flash column chromatography.

### Desymetrization of BTE-F₅Ph (3)

In order to investigate if temperature affected the product outcome in the desymmetrization of 3, we attemped the desymmetrization at different temperatures. Using one mole equivalent of hexylamine, 9 was made in 50% yield at 4° C. (Scheme 4 and Figure S20&21), and this yield remained 50% when the reaction was run at −78° C. Interstingly, the producing the disubstituted derivative 10 (Scheme 4 and Figure

Scheme 3. Desymmetrization of synthon 3. A) Desymmetrization was tolerant to aliphatic amines such as hexylamine
(C₆H₁₃), dodecylamine (C₁₂H₂₅), and methyl norbornene (CH₂—Nb). B) Reported % yield of BMA and BDA molecules
formed during a reaction, derived from ¹H NMR integration analysis.

| (A) | 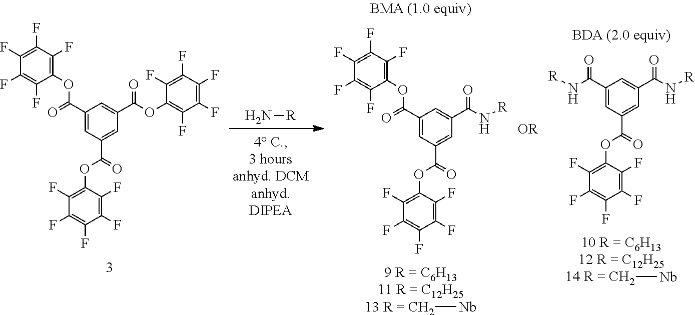 | | | |
|---|---|---|---|---|
| (B) | | Amine nucleophile (R) | | |
| | | Hexyl | Dodecyl | |
| | | (C₆H₁₃) | (C₁₂H₂₅) | CH₂—Nb |
| | BMA (%)^{a} | 48 (7) | 39 (11) | 50 (13) |
| | BDA (%)^{b} | 53 (10) | 56 (12) | 70 (14) |

S22&23) from two equivalents of amine resulted in 53% and 65% yield at 4° C. and −78° C., respectively, showing a small temperature influence on the second aminolysis. In these test reactions, we observed that only a small (5-10%) decrease in the yield after separation using silica gel flash column, resulting in a isolated yield of 40% (9) and 48% (10).

In order to test the applicability of the reaction methodology to different amine based neuclopehiles/side-arms, we attempted a few different amines. Dodecylamine, resulted in a a maximum 39% yield of 11 (monosubstituted derivative, 1 equiv amine, Scheme 3) and 56% yield of 12 (disubstituted derivative, 2 equiv amine, Scheme 3) at 4° C. (Scheme 3). There was little change in produce profile when running at −78° C., and the isolated yields again showed the stability of the activated ester to handling and purification (e.g. 49% isolated yield for 12). The desymmetrized synthons (9, 10, 11 and 12) were found to be stable over months under an inert and dry atmosphere in a desiccator at room temperature, indicating the potential for storage and resumption of synthetic pathways towards multifunctional BTAs.

After successful desymmetrization using simple hydrophobic sidearms, we next explored more functional side arms. Monosubstituted and disubstituted derivatives with 5-Norbornene-2-methylamine (5Nb-2MA,. Scheme 3) were targeted using one and two-mole equivalents to produce 13 and 14, respectively. Conviently, 13 and 14 were produced in 50% and 70% yield (via ¹H NMR integration). Going further, we explored if an amine nucleophile would show selectivity over a hydroxyl nucleophile when in competition for the activated ester. To investigate this, 3 was desymmetrized using one equivalent of 6-amino-1-hexanol and we find that the amine selectively acted as a nucleophile over the hydroxyl. ¹H NMR analysis showed that 40% of the molecules were monosubstituted, and no traces of the hydroxyl substituted core were observed

### Multifunctional BTA Derivatives Synthesis:

With the confidence that we could install different sidearms and functionalities on BTA using this activated ester methodology, we moved to create BTA derivatives with different side arms. Monosubstituted 11 was utilized and desymmetrized further, producing 15 with one dodecyl and one hexyl sidearm (Scheme 4). This reaction resulted in 60% yield based on ¹H NMR analysis and 50% isolated yield. Next, we created a molecule with one hydrophobic sidearm and one reactive functionality (norbornene), which could later be utilized for thiol-ene, norbornene-tetrazine, or ROMP polymerization. Front 7, 16 was made (Scheme 4) in 61% yield by ¹H NMR and 44% yield isolated. Important to note that in both of these reaction pathways the starting monosubstituted 7 and 11 are recovered around 10-15% and can be utilized in future reactions; thus, the isolated yield based on recovered starting material approaches 60%.

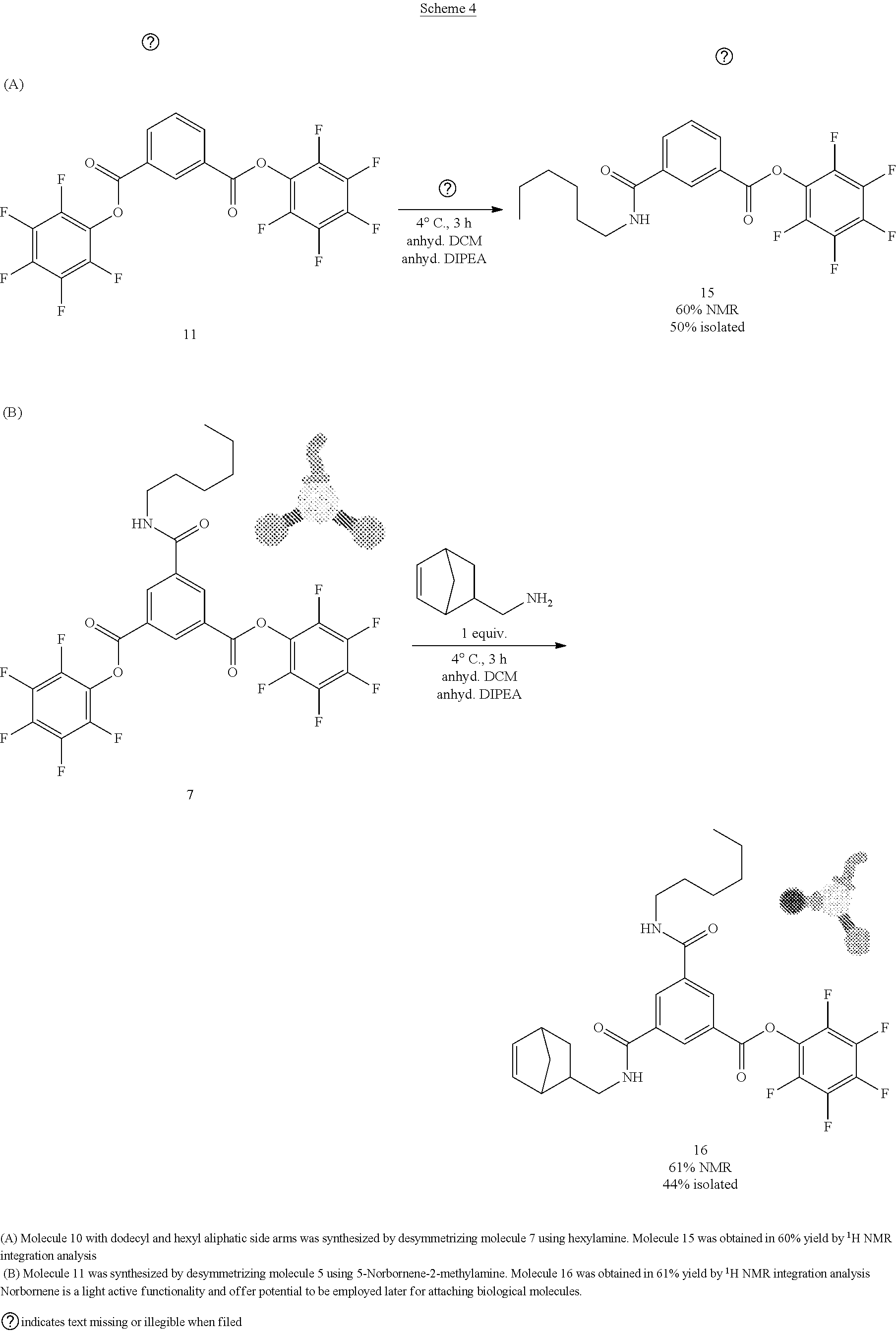

Knowing that we can create a multifunctional di-substituted derivative, next we wanted to create tri-substituted BTA derivatives. We utilized 12 with two dodecyl side arms and reacted the last activated ester to either 5Nb-2MA or 3-azido-1-propanamine to create functional BTAs 17 and 18 (Scheme 5) with orthogonally reactive handles. After an overnight (16-20 hours) reaction in DCM, ¹H NMR analysis showed 100% conversion of 12 to both 17 and 18. The isolated yield (from 12) for 17 and 18 was 87% and 85%, respectively, while the linear two step yield (starting from 3) was 42% and 40%, respectively. This shows that the high fideltiy desymmetrization can be expanded to functional handles, and potential can be utilized to attach biological molecules, probes, and effect post-assembly modifications of resultant supramolecular polymers.

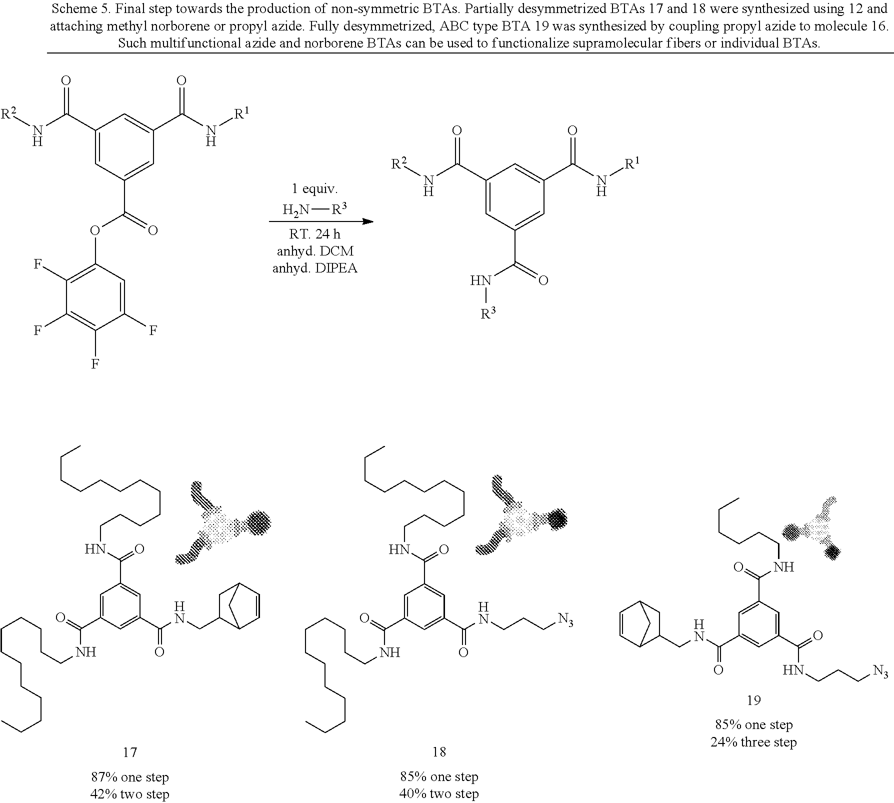

Finally, we aimed to create a fully desymmetrized ABC type BTA in order to test the full desymmetrization efficiency. In three steps, we synthesized BTA 19 (starting from 16) with a hydrophobic sidearm (hexyl), norbornene sidearm, and azide sidearm (Scheme 5) as orthogonal functionalities. After separation 19 was obtained in 85% isolated yield. Starting from symmetrical 3 to create 19 the full linear yield of the reaction was 31% by ¹H NMR and 24% by mass. This approach doubles the yield (12%) afforded by already existing desymmetrization routs to create ABC BTA 18,21 This represents the first linear and general approach towards fully desymmetrized BTAs, and this approach is envisioned to work with a wide variety of amine sidearms due to the high fidelity and mild reaction conditions.

### Molecular Design and Synthesis of Polymeric BTA (BTA-PEG20K-BTA)

Next, we moved to explore if the developed methodology could be employed for the rapid and facile creation of polymeric supramolecular macromolecules in addition to the small molecules presented above. Towards this aim, we wanted to employ our new methodology to create a small library of telechelic BTA-PEG-BTA polymers to be used as potential hydrogelators and 3D environments for cell culture. Previous work showed that a hydrophobic spacer is required to protect the BTA amides in an aqueous environment, and BTAs undergo self-assembly via hydrogen bonding and hydrophobic interactions to form long fibrils²⁰. Previous studies on telechelic BTAs have also shown that a minimum of an eight carbon hydrophobic spacer was needed for stable hydrogel formation; however, the ability to vary the outer side arm on these hydrogelator architectures has been limited by previous methodology.

In our design, we chose dodecyl as a hydrophobic spacer between BTA and PEG20K and varied the outer sidearms on the BTA. Amine end-functionalized PEG with dodecyl as an internal spacer (bisaminododecane PEG20K), was created by conjugating dodecyl diamine to PEG20K diol using carbonyldiimidazole (CDI) chemistry. A small library of BTA hydrogelators (20, 21 and 22) with C₆ sidearms (20), mixed C₆ and C₁₂ sidearms (21), C₁₂ sidearms (22) was generated by coupling bisaminododecane PEG20K to 10, 15, and 12. Important to note that this newly developed methodology not only allowed the rapid varation of outer sidearms on the BTA, but also allowed the creation of macromolecular BTA 21 with mixed outer side arms hexyl (C₆) and dodecyl (C₁₂).

All telechelic architectures were obtained with more than 95% yield, showing the high fidelity of the final pentafluorophenol ester for conjugation to macromolecules. ¹H NMR showed that all polymers are pure and the have a high degree of functionalization 81%, 67%, and 78% for hydrogelator 20, 21, and 22, respectively. GPC analysis confirms showed that all hydrogelators have weight average molecular weight around (M_{w}) around 23 kg/mol with PDI of 1.2. With this new methodology, we were able to make new telechelic BTA architectures on a multi-gram scale under two weeks, which indicates the rapid large scale capability of the designed methodology.

### Example 3

### Nb BTA Precursor Molecule Synthesis:

A dry round bottom flask was loaded with tris(perfluorophenyl)benzene-1,3,5-tricarboxylate (500 mg, 2.11 mmol, 1 equiv.) under nitrogen atmosphere and dissolved in 40 mL anhydrous dichloromethane (DCM). Subsequently solution of anhydrous N,N-diisopropylethylamine (DIPEA) (182 μL, 2.11 mmol, 1 equiv.) in 2 mL anhydrous DCM was added into the reaction flask. The recation flask was set into ice bath (4° C.). Solution of bicyclo[2.2.1]hept-5-en-2-ylmethanamine (182 μL, 1.41 mmol, 0.67 equiv.) in DCM (25 mL) was added drop wise to the reaction flask in roughly 10 minutes under nitrogen atmosphere. The reaction was stirred for 2.5 hours at 4° C. and reaction mixture was vacuum dried to remove excess solvent. Precurosor molecule (perfluorophenyl 3,5-bis((bicyclo[2.2.1]hept-5-en-2-ylmethyl) carbamoyl)benzoate) was separated by running flash column chromatography on silica gel using eluent DCM/acetonitrile (92.5/7.5) by volume. Precurosor molecule (perfluorophenyl 3,5-bis((bicyclo[2.2.1]hept-5-en-2-ylmethyl) carbamoyl)benzoate) was obtained as a white powder (202 mg) in 49% isolated yield.

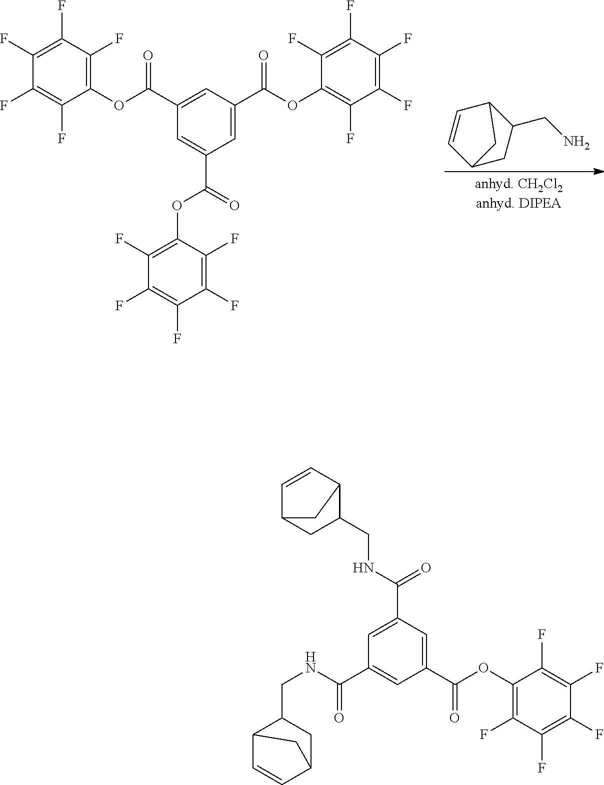 [SMILES: O=C(Oc1c(F)c(F)c(F)c(F)c1F)c1cc(C(=O)Oc2c(F)c(F)c(F)c(F)c2F)cc(C(=O)Oc2c(F)c(F)c(F)c(F)c2F)c1 ; NCC1CC2C=CC1C2 ; O=C(NCC1CC2C=CC1C2)c1cc(C(=O)NCC2CC3C=CC2C3)cc(C(=O)Oc2c(F)c(F)c(F)c(F)c2F)c1]

### PEG-CDI Synthesis:

Bishydroxy PEG (20 kg/mol) was dried via azeotropic distillation using toluene. Dried bishydroxy PEG (30 g, 3 mmol, 1equiv.) dissolve in 100 mL of anhydrous 1,4-dioxane at 37° C. under N₂ atmosphere. Vacuum dried (at 50° C. for 3 hours) carbonyl diimidazole (CDI) (0.08 g, 0.5 mmol, 5 equiv. per OH) dissolved in 30 mL of anhydrous

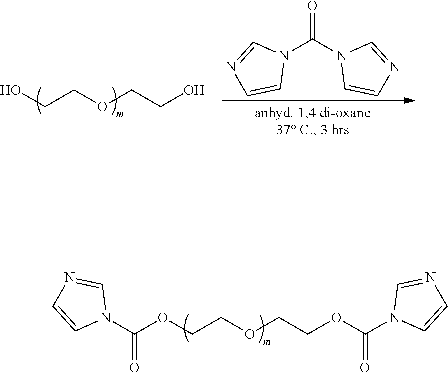

1,4-dioxane was added to the reaction flask under nitrogen inert atmosphere. The reaction mixture was stirred at 37° C. for 3 hrs. The reaction mixture was precipitated out in excess cold diethyl ether twice and dried overnight in rotavap at 40° C. The product was obtained as a white solid with a 96% (29 g) yield. 10

### PEG-Bisamino Dodecane Synthesis:

PEG-CDI (27 g, 1.34 mmol, 1 equiv.) was vacuum dried at 60° C. for 3 hours and dissolved in 270 mL anhydrous DMF. The polymer solution was added dropwise to the solution of 1,12 diaminododecane (3.5 g, 19 mmol, 14 equiv.) dissolved in 275 ml of

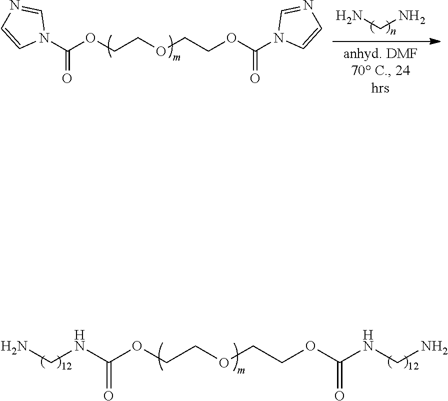

anhydrous dimethylformamide (DMF) and maintained at 70° C. The reaction mixture at 70 C was stirred for 24 hrs under nitrogen atmosphere. Reaction mixture was concentrated by removing DMF and precipitated in excess cold diethyl ether. The product was again dissolved in DCM and precipitated out in excess cold diethyl ether. The product was obtained as a white solid in 97% yield.

### Nb BTA Hydrogelator Synthesis:

In a dry round bottom flask, Precurosor molecule (perfluorophenyl 3,5-bis((bicyclo[2.2.1]hept-5-en-2-ylmethyl) carbamoyl)benzoate) (0.25 g, 0.43 mmol, 1.1equiv) was dissolved in 10 mL anhydrous DCM and DIPEA (0.076 g, 0.6 mmol, 1.5 equiv) was added into the reaction flask. Subsequently, PEG bisaminododecane (4 g, 0.4 mmol, 1 equiv) solution in anhydrous DCM (10 ml) was added dropwise to the reaction flask. The reaction mixture was stirred for 40 hrs at room temperature (˜20° C.) under nitrogen atmosphere. Excess solvent was removed in vacuo and the crude reaction mixture precipitated in excess cold diethyl ether twice and Nb BTA hydrogelator was obtained as white white powder in 98% yield (isoalted mass). Second purification was done by dialyzing sample in methanol against methanol to remove any unreacted small molecule impurities and hydrogelatro was obtained in 90% yield (isolated mass). GPC analysis showed molecular mass of 23532 g/mol with polydispersity index of 1.2.

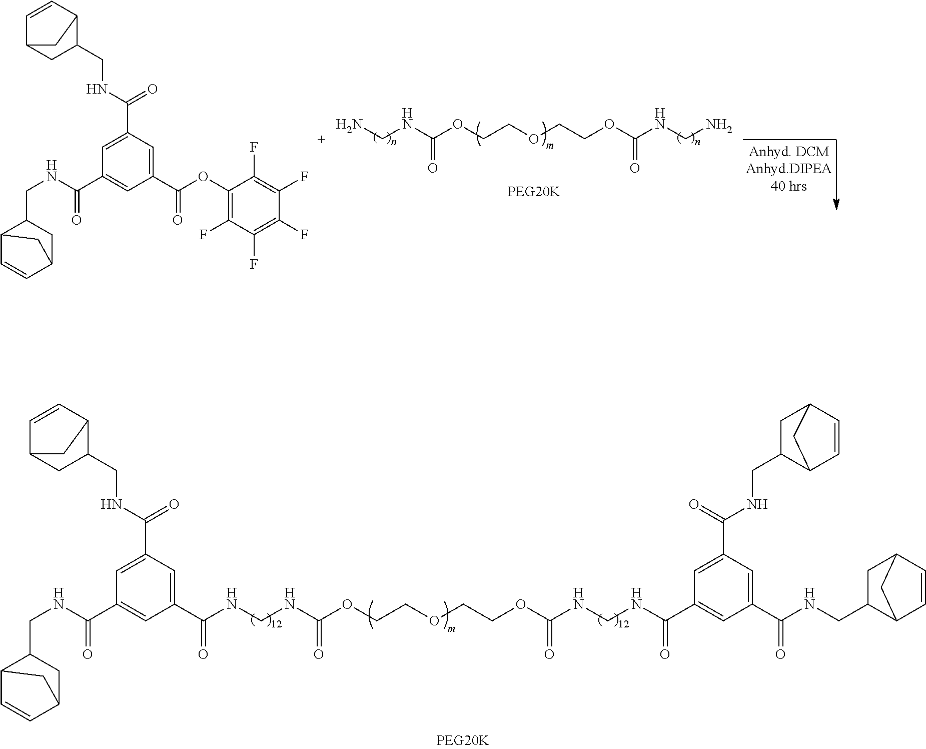

1. A compound having the formula represented by formula (Ia): 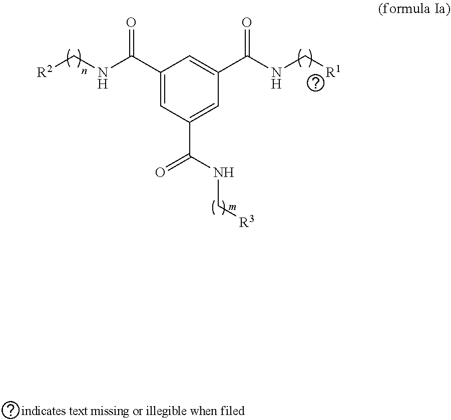
wherein:
each 1, m, and n are individually an integer between 1 to 18; and
R¹, R² and R³ are each individually selected from a polymer or a reactive group,
wherein
the polymer is represented by 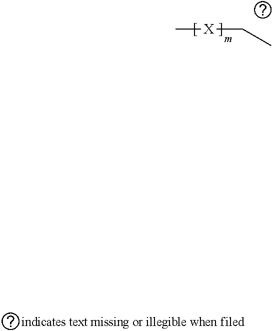
R⁴
wherein 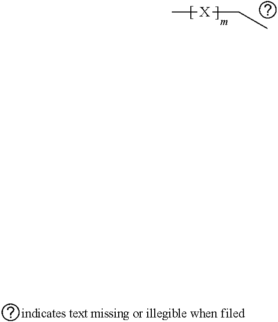
represents 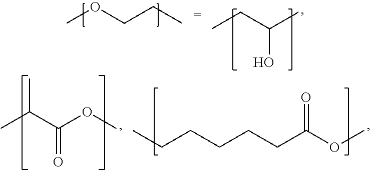
polyester co-polymers, polysaccharides, polyacrylates, polyacrylamides, polyurethanes or co-polymers thereof, wherein m is an integer from 1 to 20000 and
wherein R⁴ is a natural chain end or a reactive group, and
wherein the polymer is selected such that the compound is water soluble or water dispersible; and
wherein the reactive group is selected from the group consisting of:
a C₁-C₃₆ linear or branched alkyl or alkenyl or alkynyl or cycloalkyl, 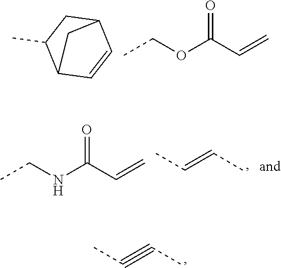
azides, alkynes, amines, oximes, hydrazides, semicarbazides, aldehydes, strained triple bonds, epoxides, malimides, furans or substitutions thereof, preferably substitutions with a linear or branched alkyl group, acrylate or acrylamide or sulphides thereof, and
wherein at least one of R¹, R² or R³ is a polymer.

2. The compound of claim 1, wherein the compound represented by formula (IVa) or formula (IVb): 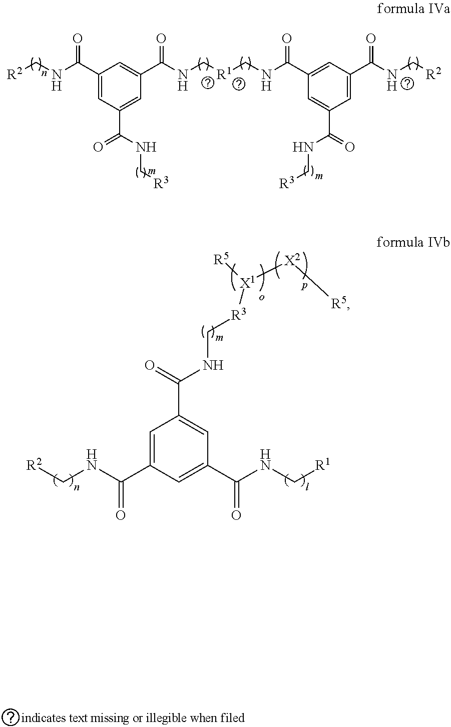
wherein X¹ and X² are each individually selected from: 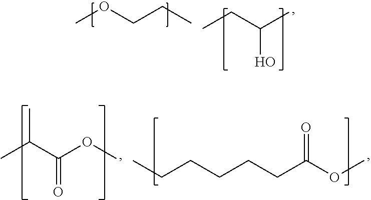
polyester co-polymers, polysaccharides, polyacrylates, polyacrylamides, polyurethanes or co-polymers thereof, wherein o and p are individually an integer from 1 to 20000,
and wherein R⁵ and R⁶ are each individually selected from H, OH, NH₂, a C₁-C₃₆ linear or branched alkyl or alkenyl or alkynyl or cycloalkyl, 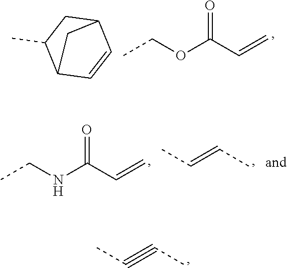
azides, alkynes, amines, oximes, hydrazides, semicarbazides, aldehydes, strained triple bonds, epoxides, malimides, furans or substitutions thereof or

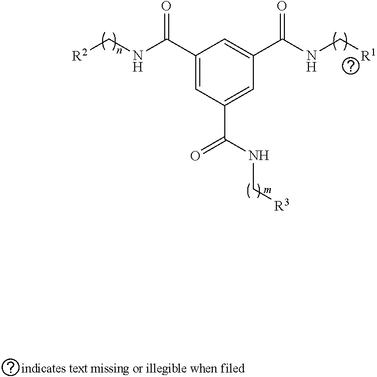

3. The compound of claim 1, wherein the compound is represented by formula (Ib): 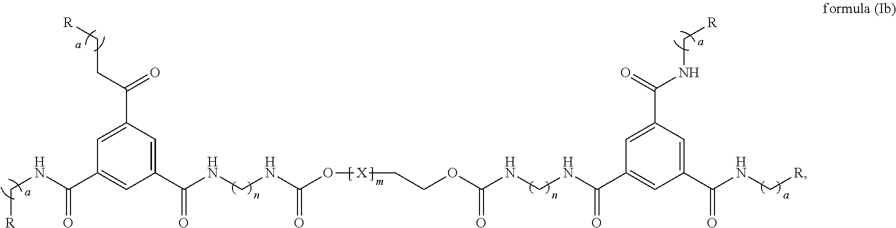
wherein:
each a is individually an integer between 1 to 18;
each n is individually an integer between 6 to 18;
wherein 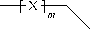
represents 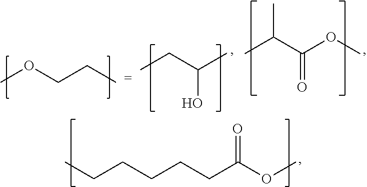
polyester co-polymers, polysaccharides, or co-polymers thereof, wherein m is an integer from 1 to 20000; and
each R is individually selected from the group consisting of:
a C₁-C₃₆ linear or branched alkyl or alkenyl or alkynyl or cycloalkyl, 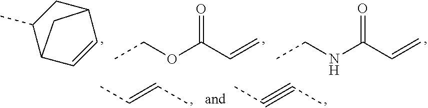
or
substitutions thereof, preferably substitutions with a linear or branched alkyl group, acrylate or acrylamide or sulphides thereof,
with the proviso that at least two, preferably at least three, more preferably all four, R are individually selected form the group consisting of: 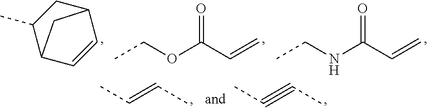
or
substitutions thereof, preferably substitutions with a linear or branched alkyl group, acrylate or acrylamide or sulphides thereof.

4. The compound according to claim 1, wherein the compound is represented by formula (IIa) or (IIb): 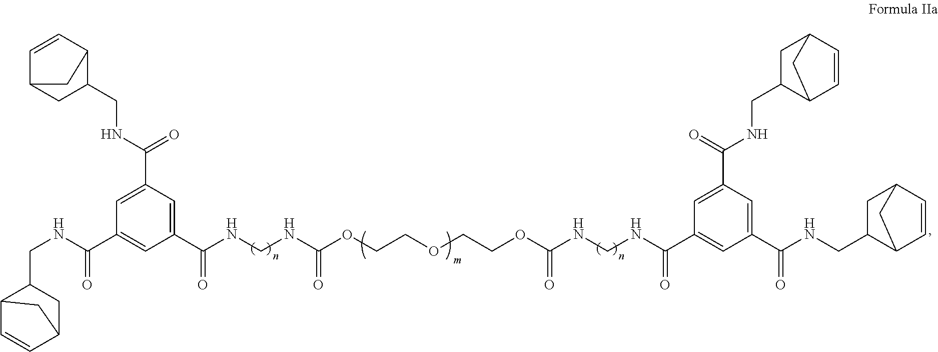
wherein:
each n is individually an integer between 6 to 18; and
m is an integer from 1 to 20000; 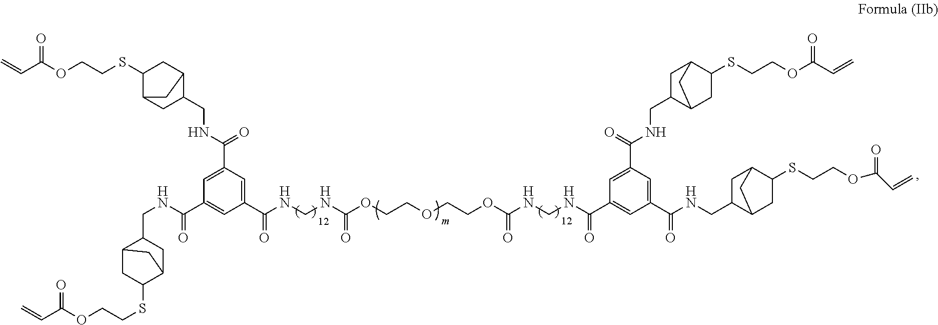
wherein:
each n is individually an integer between 6 to 18; and
m is an integer from 1 to 20000.

5. The compound of claim 1, wherein the compound is further modified with the covalent attachment of one or more peptides, antibodies, growth factors, DNA strands, glycans or combinations thereof.

6. A hydrogel comprising the compound according to claim 1.

7. The hydrogel according to claim 6, further comprising a cross-linking agent,
wherein the crosslinking agent is a compound comprising two or more reactive groups each individually selected from the group consisting of azide, alkyne, amine, oxime, hydrazide, thiol, furan, malimide, acrylate, amine and derivatives thereof,
preferably wherein the crosslinker is defined as: 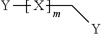
wherein 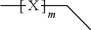
represents 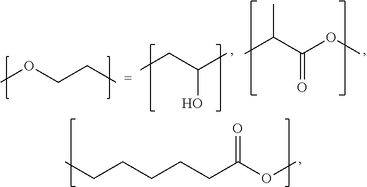
polyester co-polymers, polysaccharides, polyacrylates, polyacrylamides, polyurethanes or co-polymers thereof, wherein m is an integer from 1 to 20000, and wherein each Y is individually selected from an azide, alkyne, amine, oxime, hydrazide, thiol, furan, malimide, acrylate, amine or a derivative thereof, or
wherein the crosslinking agent is represented by formula (IIIa), formula (IIIb), formula (IIIc), formula (IIId): 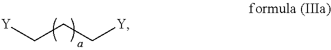
wherein a is an integer ranging from 0 to 12; 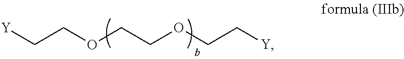
wherein b is an integer ranging from 0 to 10000; 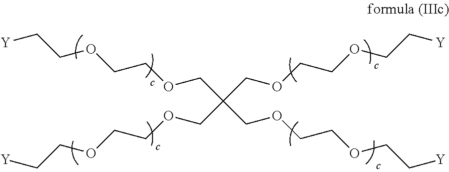
wherein each c is individually an integer ranging from 0 to 250; or 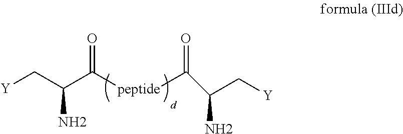
wherein 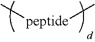
represents a peptide of 2 to 45 amino acids, preferably an enzymatically cleavable peptide; or 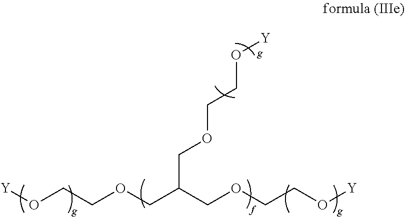
wherein f is an integer ranging from 1 to 10 and wherein each g is individually an integer ranging from 0 to 250
and wherein in the formulas IIIa, IIIb, IIIc, IIId and IIIe each Y individually is selected from an azide, alkyne, amine, oxime, hydrazide, thiol, furan, malimide, acrylate, amine or a derivative thereof.

8. The hydrogel according to claim 6 comprising between 0.25 to 30 w/v %, preferably between 1 and 10 w/v %, of the compound.

9. The hydrogel according to claim 6, wherein the molar ratio of the compound to cross-linking agent is between 10:1 and 1:4.

10. The hydrogel according to claim 6, further comprising cells, cell aggregates or organoids, preferably wherein the cells, cell aggregates or organoids are selected from chondrocytes, human umbilical vein endothelial cells (HUVECs), myocytes, neuronal cells, kidney cells, stem cells or kidney organoids.

11. A method for 3D printing a structure, the method comprising:
a. printing a composition comprising a compound having the formula represented by formula (Ia): 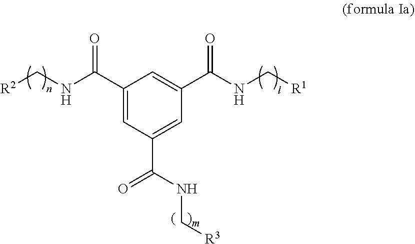
wherein:
each 1, m, and n are individually an integer between 1 to 18; and
R¹, R² and R³ are each individually selected from a polymer or a reactive group,
wherein
the polymer is represented by 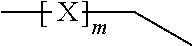
R⁴
wherein 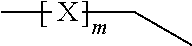
represents, 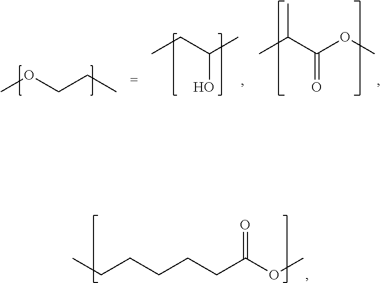
polyester co-polymers, polysaccharides, polyacrylates, polyacrylamides, polyurethanes or co-polymers thereof, wherein m is an integer from 1 to 20000 and
wherein R⁴ is a natural chain end or a reactive group, and
wherein the polymer is selected such that the compound is water soluble or water dispersible; and
wherein the reactive group is selected from the group consisting of:
a C₁-C₃₆ linear or branched alkyl or alkenyl or alkynyl or cycloalkyl, 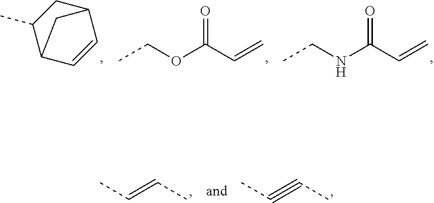
azides, alkynes, amines, oximes, hydrazides, semicarbazides, aldehydes, strained triple bonds, epoxides, malimides, furans or substitutions thereof, preferably substitutions with a linear or branched alkyl group, acrylate or acrylamide or sulfides thereof, and
wherein at least one of R¹, R² or R³ is a reactive group.

12. The method according to claim 11, wherein the composition further comprises a cross-linking agent wherein the crosslinking agent is a compound comprising two or more reactive groups each individually selected from the group consisting of azide, alkyne, amine, oxime, hydrazide, thiol, furan, malimide, acrylate, amine and derivatives thereof, preferably wherein the crosslinker is defined as: 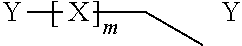
wherein 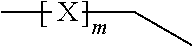
represents, 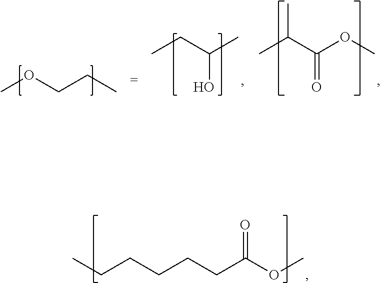
polyester co-polymers, polysaccharides, polyacrylates, polyacrylamides, polyurethanes or co-polymers thereof, wherein m is an integer from 1 to 20000, and wherein each Y is individually selected from an azide, alkyne, amine, oxime, hydrazide, thiol, furan, malimide, acrylate, amine or a derivative thereof, or wherein the crosslinking agent is represented by formula (IIIa), formula (IIIb), formula (IIIc), formula (IIId): 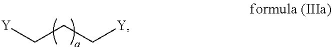
wherein a is an integer ranging from 0 to 12; 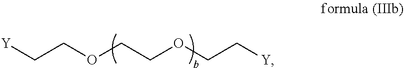
wherein b is an integer ranging from 0 to 10000; 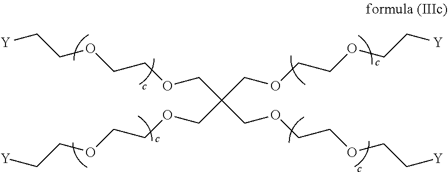
wherein each c is individually an integer ranging from 0 to 250; or 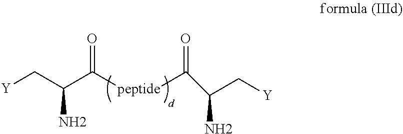
wherein 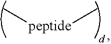
represents a peptide of 2 to 45 amino acids, preferably an enzymatically cleavable peptide; or 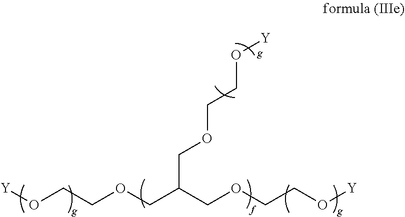
wherein f is an integer ranging from 1 to 10 and wherein each g is individually an integer ranging from 0 to 250
and wherein in the formulas IIIa, IIIb, IIIc, IIId and Ille each Y individually is selected from an azide, alkyne, amine, oxime, hydrazide, thiol, furan, malimide, acrylate, amine or a derivative thereof; and wherein the method further comprises the step:
b. cross-linking the supramolecular hydrogel structure during or after printing the structure to obtain a cross-linked hydrogel structure.

13. The method according to claim 11, wherein the composition further comprises cells, cell aggregates or organoids, preferably wherein the cells, cell aggregates or organoids are selected from chondrocytes, HUVECs, Muscle cells, Neuronal cells, kidney cells, and stem cells, kidney organoids.

14. The method according to claim 11, wherein the composition comprises between 0.25 and 30 w/v %, preferably between 1 and 10 w/v %, of the compound represented with formula I.

15. The 3D printed structure obtained or obtainable by the method according to claim 11.

16. Use of the hydrogel according to claim 6, in one or more of:
drug screening,
drug testing,
drug validation,
culturing cells or organoids,
as a model organ or model tissue.

17. The hydrogel according to claim 6, for use in a surgical method, the method comprising implanting the hydrogel or the 3D printed structure in a subject in need thereof.
